(19) United States
(12) Patent Application Publication
(10) Pub. No.: US 2017/0308677 A1
(43) Pub. Date: Oct. 26, 2017

(54) SNACKING CONTROL SYSTEM, WEARABLE DEVICE, SNACKING CONTROL METHOD, AND PROGRAM

(71) Applicant: SEIKO EPSON CORPORATION, Tokyo (JP)

(72) Inventor: Junya SHIBUYA, Shiojiri-shi (JP)

(73) Assignee: SEIKO EPSON CORPORATION, Tokyo (JP)

(21) Appl. No.: 15/491,144

(22) Filed: Apr. 19, 2017

(30) Foreign Application Priority Data
Apr. 22, 2016 (JP) 2016-085865

(51) Int. Cl. G06F 19/00; A61B 5/00; G09B 19/00; A61B 5/11

(52) U.S. Cl. CPC G06F 19/3475; G09B 19/0092; A61B 5/742; A61B 5/11; A61B 5/6824; A61B 5/4866; A61B 5/7271

(57) ABSTRACT

A snacking control system includes a snacking information acquisition section adapted to obtain snack information requested by a user, a target calorie calculation section adapted to obtain calorie information corresponding to the snack information, and calculate target calorie for the user to consume based on the snack information and the calorie information, a detection section adapted to detect activity information of the user, an activity calorie calculation section adapted to calculate activity calorie based on the activity information, a balance calorie calculation section adapted to calculate balance calorie as a difference between the activity calorie and the target calorie, and a notification section adapted to notify the user of notification information including the target calorie, the activity calorie, and the balance calorie.

### CROSS-REFERENCES TO RELATED APPLICATIONS

This application claims priority to Japanese Patent Application No. 2016-085865, filed Apr. 22, 2016, the entirety of which is herein incorporated by reference.

### BACKGROUND

### 1. Technical Field

The present invention relates to a snacking control system, a wearable device, a snacking control method, and a program.

### 2. Related Art

In the past, there has been known a system for assisting the healthcare or the diet control of a user as described in JP-2003-85289 (Document 1). This system is a system for obtaining information (images and sounds) of all of the foods and drinks including meals, snacks, and so on eaten by the user him- or herself from an individual terminal used by the user, and then providing the user with advice related to contents of foods and drinks, and health guidance based on the information.

Further, the user having need of appropriate healthcare or diet control targets on reduction in weight in many cases. In the web pages (https://www.e-healthnet.mhlw.go.jp/information/metabolic/m-03-002.html and https://www.e-healthnet.mhlw.go.jp/information/food/e-03-013.html) in the “health information site for lifestyle-related diseases prevention” by Ministry of Health, Labor and Welfare, there is also described the principle that a person facing mild through middle obesity due to metabolic syndrome targets the reduction in weight by making the energy intake by eating food and drink equal to or lower than the energy consumption by an exercise or the like due to dietary restrictions and exercise therapy. Further, in the web pages, there is described how to eat snacks as an improvement of the lifestyle for the reduction in weight. Here, since the energy intake by snacking is relatively high, there are recommended “to accurately determine a time and an amount of a nosh (snack),” “to smartly eat a snack” and so on. For example, since the energy intake (in the range of 300 through 400 kcal) by a piece of shortcake is higher than that (in the range of 240 through 270 kcal) by a bowl of rice, the proportion of the energy intake by snacking to the daily energy intake is relatively high for the user snacking on a regular basis.

However, the system described in Document 1 is a system for obtaining and analyzing the information of all of the foods and drinks eaten as meals or snacks, and is not a system focusing on snacking. Despite the fact that what the user eating the snacks on a regular basis should give attention to first is the information of the snacks, the information related to the snacks is caught up in the information of the meals. Therefore, there has been expected provision of the system placing a high value on the control related to snaking in the case in which the user eating snacks on a regular basis improves the lifestyle for the purpose of the reduction in weight.

### SUMMARY

An advantage of some aspects of the invention is to provide a system placing a high value on the control related to snaking in the case in which the user eating snacks on a regular basis improves the lifestyle for the purpose of the reduction in weight.

### Application Example 1

A snacking control system according to this application example includes a snacking information acquisition section adapted to obtain snack information requested by a user, a snacking calorie acquisition section adapted to obtain calorie information corresponding to the snack information, a target calorie calculation section adapted to calculate target calorie for the user to consume based on the snack information and the calorie information, a detection section adapted to detect activity information as information of an activity of the user, an activity calorie calculation section adapted to calculate activity calorie based on the activity information, a balance calorie calculation section adapted to calculate balance calorie as a difference between the activity calorie and the target calorie, a notification information generation section adapted to generate notification information including the target calorie, the activity calorie, and the balance calorie, and a notification section adapted to notify the user of the notification information.

According to this application example, the target calorie for the user to consume is calculated based on the snack information requested by the user and the calorie information. Further, the activity calorie can be calculated based on the activity information of the user. The notification section displays the activity calorie of the activity having been performed with respect to the target calorie, displays the balance calorie as the difference therebetween to notify the user. The user can concentrate on the activity for consuming the target calorie corresponding to the snacks. In such a manner, the user is notified of the target calorie for the user to consume and the activity calorie consumed in accordance with the activity of the user based only on the information related to the snacks as the information, instead of the control including the meals other than the snacks as in the related art. Therefore, it is possible to provide the system placing a high value on the control related to snaking in the case in which the user eating snacks on a regular basis improves the lifestyle for the purpose of the reduction in weight.

### Application Example 2

The notification information generation section generates the notification information including message information representing that a target has been achieved, in a case in which the activity calorie has become one of equal to and higher than the target calorie.

According to this application example, it is possible to notify the user of the fact that the target has been achieved.

### Application Example 3

The notification information generation section counts a number of cases in which the activity calorie has become one of equal to and larger than the target calorie, and in a case in which the number of cases has become one of equal to and larger than a predetermined number of times, the notification information generation section generates the notification information including message information representing that the user is praised.

According to this application example, the message information of praising the user is expected to have an effect of maintaining the motivation of the user in a high level.

### Application Example 4

The notification information generation section includes message information related to an activity for consuming the balance calorie in the notification information in a case in which the activity calorie is lower than the target calorie.

According to this application example, due to the message information related to the activity for consuming the balance calorie, it is possible for the user to form an image of a specific activity.

### Application Example 5

The snacking control system further includes a storage section, the snacking information acquisition section obtains a predetermined period as a period for calculating the balance calorie, the activity calorie calculation section calculates the activity calorie in the predetermined period, the balance calorie calculation section calculates the balance calorie in the predetermined period, and the storage section stores the balance calorie at an end of the predetermine period as periodic balance calorie.

According to this application example, due to the balance calorie calculated for each predetermined period, it becomes possible to verify the balance calorie every predetermined period.

### Application Example 6

The target calorie calculation section reads the periodic balance calorie from the storage section at a start of the predetermined period, and calculates the target calorie taking the periodic balance calorie into consideration.

According to this application example, due to the target calorie to which the periodic balance calorie is added, it is possible for the user to carry over the result of the predetermined period having ended to the subsequent predetermined period.

### Application Example 7

The snack information includes at least one of a name of food classification, a name of a product, and a name determined by a supplier as information related to a type, and at least one of a number, a weight, a proportion, and a volume as information related to a quantity for each type.

According to this application example, it is possible to designate the snacks with a variety of names and quantities.

### Application Example 8

The activity information includes at least one of a pulse rate, acceleration information, a heart rate, a breathing rate, and movement information.

By detecting the activity information according to this application example, the activity in the living body of the user can be figured out.

### Application Example 9

The predetermined condition includes at least one of a condition for determining an exercise state in a determination on whether or not a state is the exercise state, a condition for determining activity metabolism in a determination on whether the activity metabolism is true or basal metabolism is true, and a condition for determining an acting state in a determination on whether or not the acting state is true.

According to the predetermined condition related to this application example, the effect of consciously encouraging the exercise state, activity metabolism, and the action of the user can be expected.

### Application Example 10

The snacking control system further includes a tallying section adapted to generate total information obtained by tallying at least one of the snack information, the target calorie, the activity calorie, and the balance calorie for each evaluation period, and the notification information generation section analyzes the total information to generate the notification information expressed as a graph.

According to this application example, it is possible to look back the achievements of the snack information, the target calorie, the activity calorie, and the balance calorie.

### Application Example 11

The snacking control system further includes a second notification section adapted to notify an advisor giving advice to the user of the notification information.

According to this application example, it becomes possible for a third party (an advisor) to give advice to the user.

### Application Example 12

A wearable device according to this application example is a wearable device to be mounted to a body of a user including a snacking information acquisition section adapted to obtain snack information requested by the user, a snacking calorie acquisition section adapted to obtain calorie information corresponding to the snack information, a target calorie calculation section adapted to calculate target calorie for the user to consume based on the snack information and the calorie information, a detection section adapted to detect activity information as information of an activity of the user, an activity calorie calculation section adapted to calculate activity calorie based on the activity information, a balance calorie calculation section adapted to calculate balance calorie as a difference between the activity calorie and the target calorie, and a notification information generation section adapted to generate notification information including the target calorie, the activity calorie, and the balance calorie.

According to this application example, the target calorie for the user to consume is calculated based on the snack information requested by the user and the calorie information. Further, the activity calorie can be calculated based on the activity information of the user. The notification information includes the activity calorie with respect to the target calorie, and the balance calorie as the difference therebetween, and it is possible to notify the user of the notification information. It is possible for the user to concentrate on the activity for consuming the target calorie corresponding to the snacks in the state of wearing the wearable device. In such a manner, the user can be notified of the target calorie for the user to consume and the activity calorie consumed in accordance with the activity of the user based only on the information related to the snacks as the information, instead of the control including the meals other than the snacks as in the related art. Therefore, it is possible to provide the wearable device for controlling snacking placing a high value on snaking in the case in which the user eating snacks on a regular basis improves the lifestyle for the purpose of the reduction in weight.

### Application Example 13

A wearable device according to this application example is preferably provided with a notification section adapted to notify the user of the notification information described in Application Example 12.

### Application Example 14

A wearable device according to this application example is a wearable device to be mounted to a body of a user including a target calorie acquisition section adapted to obtain target calorie, which has been calculated based on snack information requested by the user and calorie information corresponding to the snack information for the user to consume, and which the user should consume, a detection section adapted to detect activity information as information of an activity of the user, an activity calorie calculation section adapted to calculate activity calorie based on the activity information, and a balance calorie calculation section adapted to calculate balance calorie as a difference between the activity calorie and the target calorie.

According to this application example, it becomes possible to check the activity calorie necessary to consume the target calorie corresponding to the snacks, and the balance calorie as a difference between the activity calorie and the target calorie. Therefore, it is possible to provide the wearable device for controlling snacking placing a high value on snaking in the case in which the user eating snacks on a regular basis improves the lifestyle for the purpose of the reduction in weight.

### Application Example 15

A wearable device according to this application example is preferably provided with a communication section adapted to transmit information including at least the balance calorie described in the application example 14 to an information apparatus.

### Application Example 16

A snacking control method according to this application example includes obtaining snack information requested by a user, obtaining calorie information corresponding to the snack information, calculating target calorie for the user to consume based on the snack information and the calorie information, detecting activity information as information of an activity of the user, calculating activity calorie based on the activity information, calculating balance calorie as a difference between the activity calorie and the target calorie, generating notification information including the target calorie, the activity calorie, and the balance calorie, and notifying the user of the notification information.

According to this application example, the target calorie for the user to consume is calculated based on the snack information requested by the user and the calorie information. Further, the activity calorie can be calculated based on the activity information of the user. The notifying the user displays the activity calorie of the activity having been performed with respect to the target calorie, displays the balance calorie as the difference therebetween to notify the user. The user can concentrate on the activity for consuming the target calorie corresponding to the snacks. In such a manner, the user is notified of the target calorie for the user to consume and the activity calorie consumed in accordance with the activity of the user based only on the information related to the snacks as the information, instead of the control including the meals other than the snacks as in the related art. Therefore, it is possible to provide the method placing a high value on the control related to snaking in the case in which the user eating snacks on a regular basis improves the lifestyle for the purpose of the reduction in weight.

### Application Example 17

A program according to this application example makes a computer perform obtaining snack information requested by a user, obtaining calorie information corresponding to the snack information, calculating target calorie for the user to consume based on the snack information and the calorie information, detecting activity information as information of an activity of the user, calculating activity calorie based on the activity information, calculating balance calorie as a difference between the activity calorie and the target calorie, generating notification information including the target calorie, the activity calorie, and the balance calorie, and notifying the user of the notification information.

According to this application example, the target calorie for the user to consume is calculated based on the snack information requested by the user and the calorie information. Further, the activity calorie can be calculated based on the activity information of the user. The notifying the user displays the activity calorie of the activity having been performed with respect to the target calorie, displays the balance calorie as the difference therebetween to notify the user. The user can concentrate on the activity for consuming the target calorie corresponding to the snacks. In such a manner, the user is notified of the target calorie for the user to consume and the activity calorie consumed in accordance with the activity of the user based only on the information related to the snacks as the information, instead of the control including the meals other than the snacks as in the related art. Therefore, it is possible to provide a program realizing the method placing a high value on the control related to snaking in the case in which the user eating snacks on a regular basis improves the lifestyle for the purpose of the reduction in weight.

### BRIEF DESCRIPTION OF THE DRAWINGS

The invention will be described with reference to the accompanying drawings, wherein like numbers reference like elements.

FIG. 1 is an explanatory diagram showing an outline of a snacking control system.

FIG. 2 is a block diagram showing an outline function of the snacking control system.

FIG. 3 is a diagram showing an example of an input screen.

FIG. 4 is a diagram showing an example of a notification information screen.

FIG. 5 is a diagram showing an example of the notification information screen.

FIG. 6 is a diagram showing an example of the notification information screen.

FIG. 7 is a diagram showing an example of the notification information screen.

FIG. 8 is a diagram showing a graph screen of total information.

FIG. 9 is a diagram showing a graph screen of total information.

FIG. 10 is a diagram showing a graph screen of total information.

FIG. 11 is a diagram showing a graph screen of total information.

FIG. 12 is a flowchart showing a flow of a process of snacking control.

FIG. 13 is a flowchart showing a flow of a calculation process of target calorie.

FIG. 14 is a flowchart showing a flow of a generation process of notification information.

FIG. 15 is a diagram showing an outline of an information apparatus according to a second embodiment.

FIG. 16 is an explanatory diagram showing an outline of a snacking control system according to a modified example.

### DESCRIPTION OF EXEMPLARY EMBODIMENTS

Some embodiments of the invention will hereinafter be described with reference to the accompanying drawings. It should be noted that in each of the drawings described below, the scale sizes of the sections and the screens are made different from actual ones in order to make the sections and the screens have recognizable dimensions.

### First Embodiment

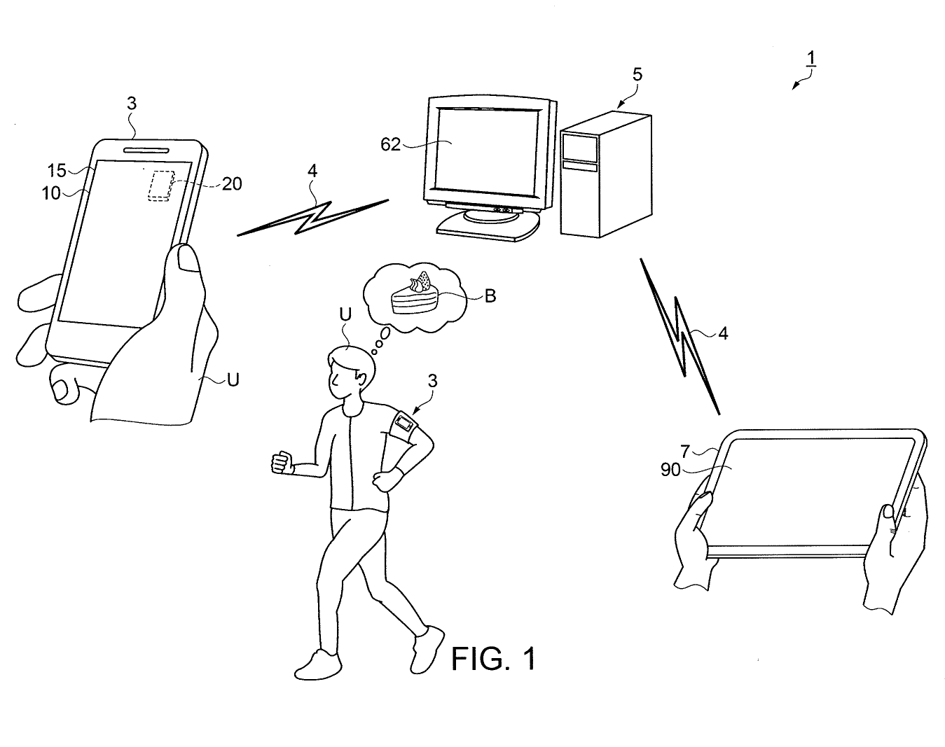

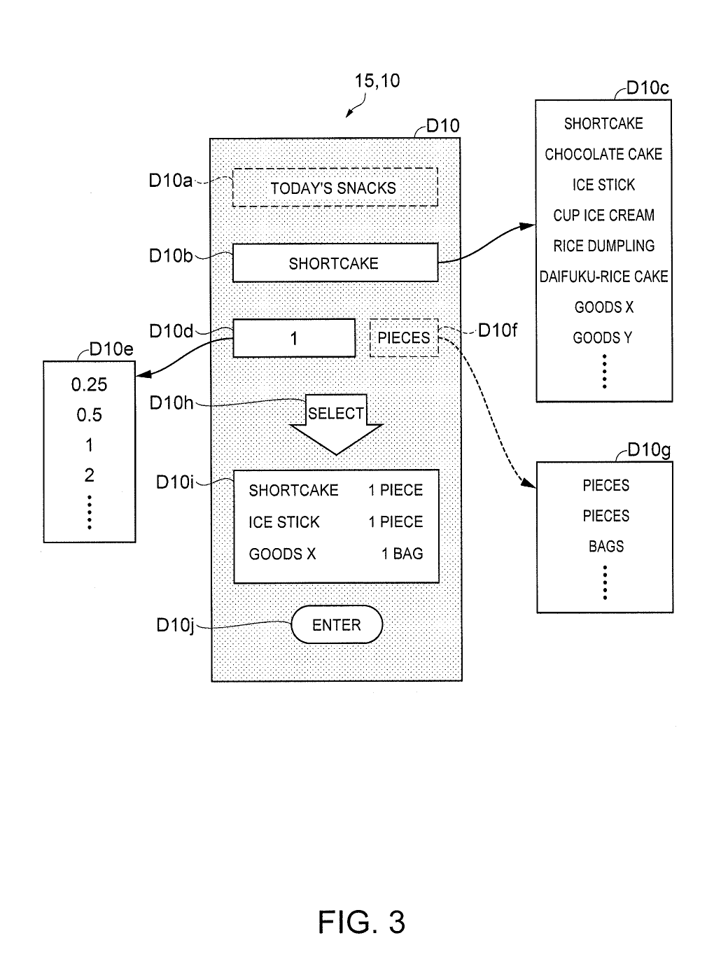

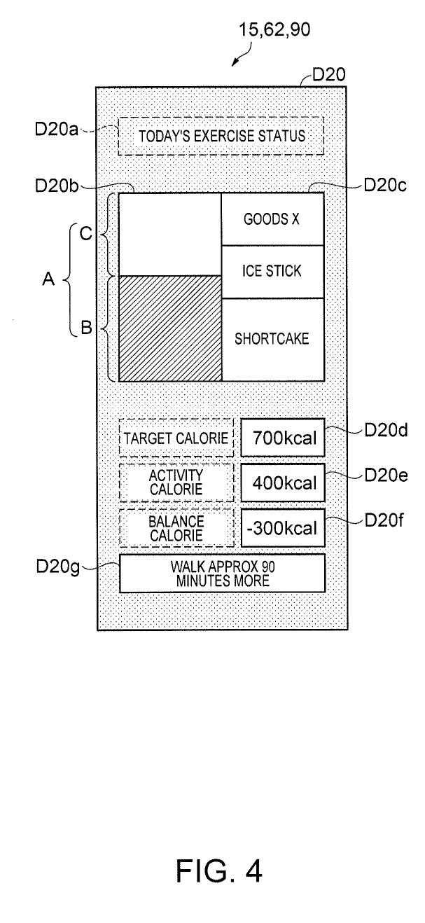

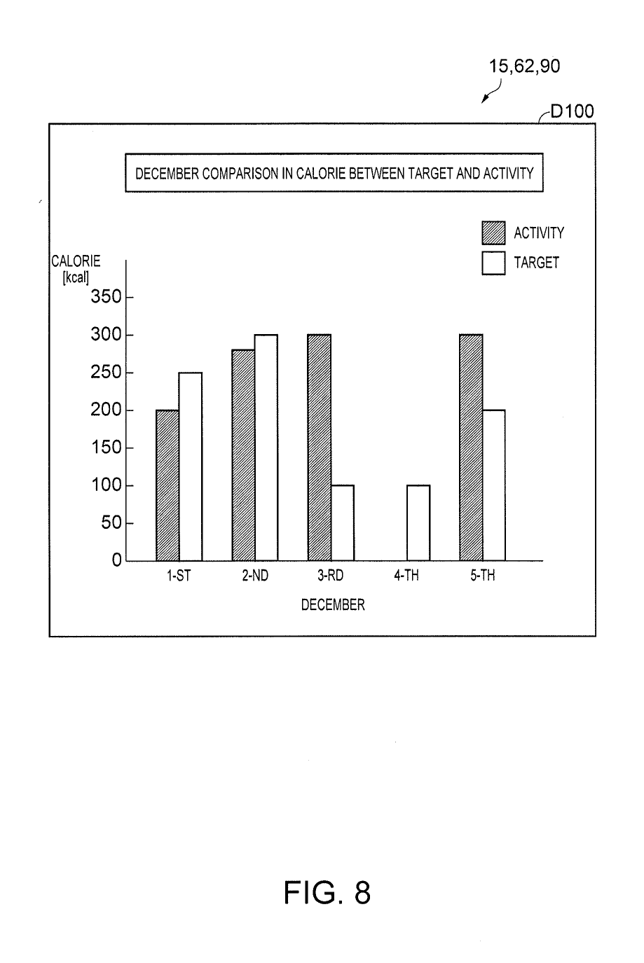

FIG. 1 is an explanatory diagram showing an outline of a snacking control system. FIG. 3 is a diagram showing an example of an input screen, FIG. 4 through FIG. 7 are each a diagram showing an example of a notification information screen, and FIG. 8 is a diagram showing a graph screen of total information.

A snacking control system 1 shown in FIG. 1 is a system used by a user U which improves the lifestyle for the purpose of the reduction in weight. The snacking control system 1 is constituted by an information apparatus 3 carried at all times by the user U, a server 5 connected to a network so as to be able to perform data communication (communication 4), an information apparatus 7 available for the user U and other users, and so on.

The information apparatus 3 is a general smartphone or portable terminal, and is provided with a web browser and an Internet communication function, and is capable of installing and then executing a web application and application software downloaded. In the information apparatus 3, when a program 59 (described later) for controlling snacking is executed, a screen D10 (FIG. 3) is displayed on a display section 15 as a liquid crystal display screen. The screen D10 is a screen for selecting “TODAY'S SNACK,” and the snack displayed in an area D10i of the screen is selected. It should be noted that on a surface of the liquid crystal display screen, a touch panel for receiving input as an input section 10 is disposed, and receives the input of these alternatives. As today's snacks, the user U plans to eat (ingest) all of “ONE PIECE OF SHORTCAKE,” “ONE ICE STICK,” and “ONE BAG OF GOODS X” displayed in the area D10i. It should be noted that the goods X is the trade name of a snack.

The user U acts carrying the information apparatus 3 at all times. As shown in FIG. 1, the information apparatus 3 is mounted so as to be wound around the left upper arm of the user U with a belt. The user U is jogging (acting) while thinking of the shortcake (an image B) as the snack selected in the area D10i of the screen D10.

The information apparatus 3 incorporates a detection section 20. The detection section 20 is, for example, an acceleration sensor 21 (described later), and detects acceleration information changing with the body motion of the user U. In the information apparatus 3, an amount of metabolic heat (hereinafter referred to as activity calorie) metabolized while exercising is calculated based on the acceleration information. A screen D20 (FIG. 4) is a display screen appearing after the user U has performed the exercise corresponding to the activity calorie of 400 kcal while carrying the information apparatus 3. In the area of a graph D20c, there are displayed the snacks selected in the area D10i of the screen D10. The caloric value (700 kcal) obtained by summing up the food caloric values (hereinafter referred to as snacking calorie) of the snacks is displayed in an area D20d as a caloric value of the target calorie. It should be noted that the snacking calorie corresponds to calorie information. In the information apparatus 3, the snacking calories of the snacks planned to be “eaten (accumulated in the body) as snacks today” are summed up, and then the result is set as the target calorie to be “consumed (burnt) by exercise today.”

The user U makes a plan assuming that the snacks displayed in the area D10i of the screen D10 are planned to be eaten today, and then performs the exercise for consuming the target calorie while visualizing a scene of eating the snack of the image B. The actual achievement of the activity calorie consumed by the exercise can be checked on the screen D20 at any time. In such a manner, in the information apparatus 3, the plan of eating the snacks and the actual achievement of the exercise performed for consuming the caloric value corresponding to the snacks can be controlled.

In the server 5, the information such as the target calorie and the activity calorie is received from the information apparatus 3, and is then analyzed. The information thus analyzed is displayed on a display 62. Further, the information is transmitted to the information apparatus 7, and in the information apparatus 7, the information related to the snacks planned to be eaten by the user U, and the information of the target calorie and the activity calorie are obtained, and are then displayed on a display section 90. It is possible for the user using the information apparatus 7 to advise on how to eat the snacks and the activity as an advisor to the user U.

As described above, the snacking control system 1 is made as a system placing a high value on the control related to snaking. The configuration of the snacking control system 1 capable of achieving such an advantage will hereinafter be described in detail.

### Configuration of Snacking Control System

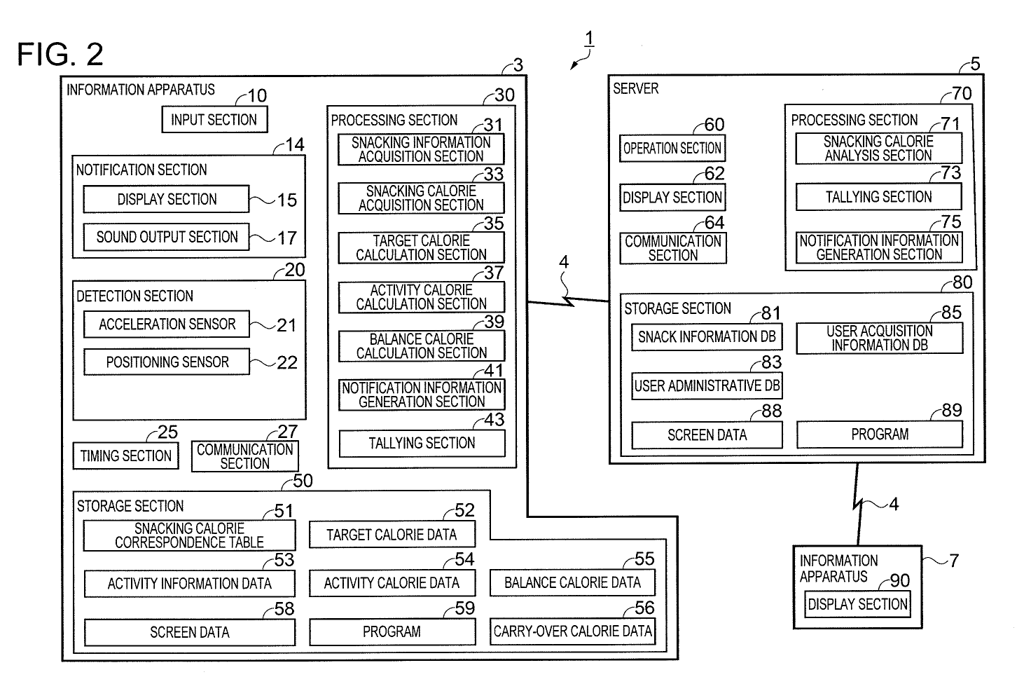

FIG. 2 is a block diagram showing an outline function of the snacking control system. The snacking control system 1 is constituted by the information apparatus 3, the server 5, the information apparatus 7 and so on.

As described above, the information apparatus 3 is a general smartphone or cellular phone. Further, it is sufficient for the information apparatus 3 to be a portable apparatus, and it is also possible for the information apparatus 3 to be a tablet terminal, an HMD (head-mounted display), a notebook PC (personal computer), a PDA (personal digital assistant) or the like.

The information apparatus 3 is constituted by the input section 10, a notification section 14, the detection section 20, a timing section 25, a communication section 27, a processing section 30, a storage section 50 and so on.

The input section 10 is an input device configured including a touch panel covering a display surface of the display section 15, and detects an input operation by the user and then output the result to the processing section 30. The touch panel outputs position information corresponding to a button widget, input area and so on displayed on the display section 15 to the processing section 30. In the processing section 30, there is performed a process corresponding to the input operation based on the information displayed on the display section 15 and the position information obtained from the touch panel.

The notification section 14 is constituted by the display section 15, a sound output section 17 and so on.

The display section 15 is a display device configured including a liquid crystal display (LCD) or the like, and displaying a variety of types of notification information based on a display signal input from the processing section 30. Image data such as the screen D20 generated by the processing section 30 is output to the display section 15.

The sound output section 17 is a sound output device configured including a speaker, a piezoelectric vibrator or the like, and outputting a variety of types of notification information based on an output signal input from the processing section 30. It should be noted that the sound output section 17 can also be provided with an earphone jack or a near-field wireless communication function, and in such a case, it is also possible for the sound output section 17 to transmit a sound output signal to an earphone or a headphone worn by the user.

### Detection Section

The detection section 20 is constituted by the acceleration sensor 21, a positioning sensor 22 and so on.

The acceleration sensor 21 is preferably an acceleration sensor unit capable of detecting the accelerations in three-axis directions roughly perpendicular to each other, and outputs acceleration information in the three-axis directions thus detected to the processing section 30. The acceleration sensor 21 measures an acceleration variation in each of the axes at sampling intervals. As a preferable example, the sampling frequency is set to be equal to or higher than 16 Hz. The acceleration sensor 21 detects the motion of the user, performs amplification, shaping and A/D conversion on the acceleration signal thus detected in an amplifier circuit, a waveform shaping circuit and an A/D conversion circuit (all not shown), and then outputs the result as the acceleration information. The acceleration information corresponding to the three axes is stored in the storage section 50 as a part of activity information data 53 by the processing section 30.

It should be noted that the acceleration sensor 21 is assumed to be the sensor unit having a triaxial acceleration sensor, but it is sufficient for the acceleration sensor 21 to be a sensor unit having an acceleration sensor with at least two axes. It is also possible to be provided with an acceleration sensor with two axes roughly perpendicular to each other, or to be provided with an acceleration sensor with four or more axes three-dimensionally crossing each other.

The positioning sensor 22 is configured including an antenna section for receiving a satellite signal from a positioning GNSS (global navigation satellite system) satellite, a front-end section, and a positioning information calculation section (all not shown), and outputs the positioning information of the information apparatus 3 to the processing section 30. The positioning information includes latitude, longitude, altitude and so on, and is output as movement information every unit time (e.g., every 1 second). The movement information is stored in the storage section 50 as apart of the activity information data 53 by the processing section 30.

It should be noted that it is also possible to adopt a configuration in which the detection section 20 is provided with a gyro sensor. The gyro sensor detects the angular velocity taking the three axes roughly perpendicular to each other as the central axes, and then outputs angular velocity information. The angular velocity information is stored in the storage section 50 as a part of the activity information data 53 by the processing section 30.

It should be noted that the detection section 20 is not limited to the acceleration sensor 21, the positioning sensor 22 and so on incorporated in the main body of the information apparatus 3, but can also be constituted by a sensor device capable of communicating with, and separated from the information apparatus 3. The detection section can also be, for example, a sensor device provided with pulse sensors 123, 223 (described later in detail) mounted on a skin surface of a human body to detect the pulse rate, a sensor device provided with a heartbeat sensor making a plurality of electrodes have contact with a skin surface of a human body to detect the hart rate, a sensor device provided with a sound measurement sensor having a heart sound microphone to measure a heartbeat sound and a breath sound of a human body, or the like. The information of the pulse rate, the heart rate, or the breath sound detected by such sensor devices is transmitted to the processing section 30 via the communication section 27, and is then stored as a part of the activity information data 53 in the storage section 50.

The timing section 25 is a real-time clock, and has timing functions such as generation of sampling time for the acceleration information and the positioning information detection, a calendar function, a timing function and a stopwatch function. Further, there is output time and date information such as a period for referring to the target calorie, the activity calorie, balance calorie, and carry-over calorie described later.

The communication section 27 is a communication adapter provided with a wireless communication adapter for cellular phone communication for communicating with the server 5 via the communication 4, a wireless LAN (local area network) and so on. Further, the communication section 27 is provided with a near-field communication adapter to be connected to a separate device such as the sensor device (the detection section 20) described above so as to be able to communicate with the separate device. It should be noted that the communication section 27 can be a communication adapter including a physical communication terminal or a wireless terminal and having a common communication protocol, and in such a case, the both parties are connected to each other via a cable or radio to transmit and receive a variety of types of data.

The processing section 30 is a control device and an arithmetic device configured including a processor such as a central processing unit (CPU), and integrally controlling the sections of the information apparatus 3. The processing section 30 realizes a variety of functions of the information apparatus 3 in accordance with a variety of programs (not shown) including the program 59 stored in the storage section 50. It should be noted that the processing section 30 corresponds to a computer.

The processing section 30 executes the program 59 stored in the storage 50 to realize the functions of respective functional sections, namely a snacking information acquisition section 31, a snacking calorie acquisition section 33, a target calorie calculation section 35, an activity calorie calculation section 37, a balance calorie calculation section 39, a notification information generation section 41, and a tallying section 43. It should be noted that these functional sections are illustrative only, and all of the functional sections are not necessarily essential constituents.

### Snacking Information Acquisition Section

The snacking information acquisition section 31 is a functional section for controlling the input section 10 to obtain a variety of types of information from the user, and obtains a variety of types of configuration information related to the snacking control, snack information requested by the user, and so on.

The variety of types of configuration information includes information of a predetermined period related to the period for controlling the snacking, and information (carry-over ON/OFF information) representing whether or not the balance calorie in the previous predetermined period is carried over every predetermined period. In the snacking information acquisition section 31, the input section 10 is controlled to make the user select the predetermined period from the alternatives of “one day,” “one week,” and so on, and select the carry-over ON/OFF information from the alternatives of “ON” and “OFF.” The setting contents (not shown) of the predetermined period and the carry-over ON/OFF information thus obtained are stored in the storage section 50.

It should be noted that the predetermined period is a period for calculating the balance calorie described later.

The snacking information acquisition section 31 obtains the snack information requested by the user. In detail, the snacking information acquisition section 31 generates the input screen for prompting input of the snack information, and then outputs the input screen to the display section 15. The snacking information acquisition section 31 controls the input section 10 to obtain the information thus selected. The snack information includes a type of the snack thus requested, and an amount of the snack.

The screen D10 shown in FIG. 3 is an example of the input screen, and for receiving the input of the type of the snack and the amount of the snack. In the example of the screen D10, “one day” is set as the predetermined period.

In the screen D10, “TODAY'S SNACKS” is displayed in the area D10a. The term “today” is an example representing the predetermined period, and represents that the predetermined period in this case is “one day” from 00:00:00 to 23:59:59 of the day that the screen D10 displays. In the area D10b, there is displayed a candidate of the snack, and in the alternatives D10c, there are displayed the types of the snacks which can be selected. As the types of the snacks, there can be cited names in the food classification, names of commercial goods, names determined by food suppliers, and so on. When the user selects the snack the user wants to eat or drink from the alternatives D10c, the snack is displayed in the area D10b. In the alternatives D10c, there are shown the GOODS X and the GOODS Y as the alternatives. These are examples of the names of the commercial goods. As described above, the names of the commercial goods can also be selected as the snack. The names determined by the food suppliers are names of foods and drinks which are not commercially available, such as “homemade cake by A.” Further, in the case in which the snack the user wants does not exist in the alternatives D10c, it is possible for the user to directly input the name of the snack in the area D10b. The area D10d is an area for inputting an amount of the snack thus selected, and the alternatives D10e are numerical values of the snack. The area D10f shows the unit of the amount of the snack, and the unit of the candidate of the snack displayed in the area D10b is selected from the types of the units shown in the alternatives D10g, and is then displayed in the area D10f. As the unit of the snack, the number, the weight, the proportion, the volume, and so on can also be selected. The proportion and the volume are often used in the case in which the type of the snack is a drink, and are the proportion to 1 goh (Japanese unit of volume equivalent to 0.18 litter) such as 0.5 goh of sake, a volume such as litter in 1 litter of milk, and so on. The button D10h is the button widget capable of performing input, and when the button is held down, the candidate of the snack is displayed in the area 10Di.

The snack and the amount displayed in the area D10i are the snack information planned to be eaten or drunk in the predetermined period as the “TODAY'S SNACKS.” The button D10j is used for deciding the snacks and the amounts of the snacks displayed in the area D10i.

In the snacking information acquisition section 31, when the snack information has been obtained in such a manner, the snack information thus obtained is delivered to the snacking calorie acquisition section 33.

### Snacking Calorie Acquisition Section

The snacking calorie acquisition section 33 obtains the snacking calorie representing the food caloric values of the snack for each of the types of the snacks. In detail, the snacking calorie acquisition section 33 obtains the corresponding snacking calorie for each of the snacks with reference to a snacking calorie correspondence table 51 stored in the storage section 50. In the snacking calorie correspondence table 51, the information of the unit of the amount of the snack and the food caloric value per unit amount is stored for each of the types of the snacks. In the snacking calorie acquisition section 33, the information of the snacking calorie per unit amount is obtained from the snacking calorie correspondence table 51 for each of the snacks in the snack information delivered from the snacking information acquisition section 31.

Further, in the case in which the snack information thus delivered includes an item not included in the snacking calorie correspondence table 51, the snacking calorie acquisition section 33 controls the communication section 27 to establish the communication 4 with the server 5, and thus, receives the information of the snacking calorie corresponding to the snack information from the server 5. In the server 5, the information of a variety of types of snacks is accumulated in a snack information DB 81 (described later), and the information of those snacks is updated with a new content in each case. In the snacking calorie acquisition section 33, when the information related to the new snack and the snacking calorie per unit amount of that snack are obtained from the server 5, the information and the snacking calorie thus obtained are additionally written to the snacking calorie correspondence table 51.

The snacking calorie acquisition section 33 delivers the snacking calorie per unit amount for each of the snacks thus obtained and the amount of each of the snacks to the target calorie calculation section 35.

### Target Calorie Calculation Section

As the target calorie, the target calorie calculation section 35 calculates an amount of the metabolic heat necessary for consuming the food caloric value calculated from the snack information (the type and the amount of each of the snacks) and the snacking calorie (the snacking calorie per unit amount). Then, the target calorie calculation section 35 stores the target calorie thus calculated in the storage section 50 as target calorie data 52. In detail, in the target calorie calculation section 35, the amount of the snack and the snacking calorie per unit amount are multiplied by each other for each of the types of the snacks delivered from the snacking calorie acquisition section 33, then the results are added to each other to obtain the sum of the food caloric values corresponding to the snacks the user wants. The sum of the food caloric value is calculated as the target calorie.

It is also possible for the target calorie calculation section 35 to refer to the carry-over ON/OFF information obtained by the snacking information acquisition section 31. If the carry-over ON/OFF information represents “OFF,” the sum of the food caloric values becomes the target calorie as described above, and if the carry-over ON/OFF information represents “ON,” the carry-over calorie data 56 as the balance calorie in the previous predetermined period is added. For example, if the previous balance calorie is “−200 kcal, ” the result that “the activity calorie is 200 kcal smaller than the target calorie” is obtained, and if the carry-over ON/OFF information represents “ON,” 200 kcal is added to the target calorie. In other words, this corresponds to the setting in which the shortage of the exercise quantity in the previous time (e.g., yesterday) is recovered this time (today). Further, for example, if the previous balance calorie is “+200 kcal,” since the amount of the metabolic heat in the previous exercise is subtracted from the target calorie this time, it is possible to set the target calorie in such a manner that exercise is reduced today as much as the exercise performed yesterday.

It should be noted that although it is assumed that the sum of the food caloric values is set to the target calorie, it is also possible to obtain the target calorie by multiplying the sum of the food caloric values by a predetermined ratio or a predetermined conditional expression. Further, it is also possible to change the predetermined ratio, the predetermined conditional expression, and so on by the type of the snack.

### Activity Calorie Calculation Section

The activity calorie calculation section 37 obtains the activity information detected by the detection section 20, and then calculates the activity calorie obtained by accumulating the amount of metabolic heat consumed in accordance with the activity satisfying predetermined conditions. The activity calorie calculation section 37 stores the activity calorie thus calculated in the storage section 50 as activity calorie data 54.

In detail, the information such as the acceleration information, the movement information, the pulse rate and the heart rate is stored every unit time as time-series data in the activity information data 53 by the detection section 20. In the activity calorie calculation section 37, such information is read, and is then compared with the predetermined conditions. The predetermined conditions are for determining whether or not the state is an exercise state (“no” means a normal life state) (hereinafter referred to as a first condition), whether the state is an activity metabolism state or a basal metabolism state (hereinafter referred to as a second condition), and whether or not the state is an acting state (“no” means a state with little action) (hereinafter referred to as a third condition), and if the state is the exercise state, the activity metabolism state, and the acting state, it is determined that the predetermined conditions are satisfied.

Each of the predetermined conditions will specifically be described.

For example, a method of determining the behavioral pattern of the user using the acceleration information is described in JP-A-2014-212915. According to this method, “rest,” “life action,” “walking,” “running,” “strength training,” “exercising,” “bicycle,” “other exercises,” and so on can accurately be determined based on the acceleration information. Among these states, regarding the first condition and the second condition, “walking,” “running,” “strength training,” “exercising,” “bicycle,” and “other exercises” are in the exercise state and the activity metabolism state, and are therefore determined to satisfy the predetermined conditions. Regarding the third condition, “life action,” “walking,” “running,” “strength training,” “exercising,” “bicycle,” and “other exercises” are in the acting state, and are therefore determined to satisfy the predetermined condition.

It should be noted that according to JP-A-2014-212915, the behavior determination can more accurately be achieved using the information such as the movement information and the pulse rate, and therefore, the information obtained by the detection section 20 can effectively be used for the accurate determination.

The activity calorie calculation section 37 calculates an amount of the metabolic heat (the calorie consumption) consumed in the state satisfying the predetermined conditions, then accumulates the results, and then obtains the result as the activity calorie. An amount of the metabolic heat to be consumed can be calculated using a method of multiplying the exercise load (e.g., METs as an index of the exercise intensity) of the behavioral pattern obtained by determining the activity of the user as described above and the duration of the exercise by each other. Further, it is also possible to calculate an amount of the metabolic heat to be consumed using the body information such as the age, the sex, the height and the weight of the user as a coefficient of an amount of the metabolic heat.

Further, as a method of using the information such as the pulse rate and the heart rate, the exercise intensity is calculated using the correlative relationship appearing between the variation in pulse rate or heart rate and the variation in oxygen uptake, and then the activity calorie corresponding to the exercise intensity is calculated. The correlative relationship is defined in accordance with the body information such as age, sex, and weight, and is stored in the storage section 50 in advance. It should be noted that as the calorie calculation method, it is possible to adopt a method of using an output signal from a gyro sensor. In detail, it is possible to calculate the exercise intensity based on the variation and the intensity of the acceleration information due to the body motion output from the acceleration sensor and the gyro sensor, and then calculate the activity calorie corresponding to the exercise intensity.

### Balance Calorie Calculation Section

The balance calorie calculation section 39 calculates the balance calorie as a difference between the activity calorie and the target calorie. In detail, the balance calorie calculation section 39 reads the activity calorie data 54 and the target calorie data 52 stored in the storage section 50, and then subtracts the target calorie from the activity calorie to obtain the balance calorie. The balance calorie is stored in the storage section 50 as balance calorie data 55. The balance calorie can also be a value obtained by subtracting the activity calorie from the target calorie. In this case, since the sign of the balance calorie is reversed, it is sufficient to take the reversion of the sign into consideration in each of other functional sections referring to the balance calorie.

It should be noted that in the activity calorie calculation section 37, by performing the calculation taking the information inherent in the user such as the age, the sex, the weight, the basic physical fitness information of the user into consideration in the calculation of the calorie consumption and the determination of the behavioral pattern of the user, the information higher in accuracy can be calculated. It is also possible to adopt a configuration in which such information inherent in the user is set in the information apparatus 3 by the user in advance, or received from the server 5 by controlling the communication section 27.

Further, when the predetermined period ends, the balance calorie calculation section 39 stores the balance calorie at that moment in the storage section 50 as the carry-over calorie data 56. It should be noted that the carry-over calorie data 56 corresponds to periodic balance calorie.

### Notification Information Generation Section

The notification information generation section 41 generates the notification information screen including the target calorie, the activity calorie, the balance calorie, and message information to the user.

The display content of the notification information screen including the message information to be generated differs by the values of the target calorie, the activity calorie and the balance calorie, and the content of the carry-over ON/OFF information. An example of the notification information screen shown in FIG. 4 through FIG. 7 will be described using the drawings.

The notification information screen shown in FIG. 4 is a screen (the screen D20) displayed in the state in which the carry-over ON/OFF information is set to “OFF,” and the balance calorie has a negative value. Further, the snacks the user wants are “SHORTCAKE,” “ICE STICK,” and “GOODS X.”

In the screen D20, “TODAY'S EXERCISE STATUS” is displayed in the area D20a. The term “today” in “TODAY'S EXERCISE STATUS” represents that the predetermined period is one day. In the areas D20d, D20e, and D20f, there are shown that the target calorie is “700 kcal” (the area D20d), that the activity calorie is “400 kcal” (the area D20e), and that the balance calorie is “−300 kcal” (the area D20f), respectively. The balance calorie is a value obtained by subtracting the target calorie from the activity calorie.

The graph D20b is a band graph, and shows that the hatched part of the zone B corresponds to the activity calorie of the exercise actually performed with respect to the target calorie in the zone A. As specific numerical values, the zone A represents the target calorie (700 kcal, i.e., 100%), the zone B represents the activity calorie (400 kcal/700 kcal, i.e., 57%), and the zone C represents the balance calorie (300 kcal/700 kcal, i.e., 43%).

The graph D20c is a band graph, and is a graph shown so as to show the proportions of the snacking calories of the snacks the user wants. The height direction of the band graph corresponds to 100%, which coincides with 100% of the target calorie in the graph D20b. The shortcake, the ice stick, and the goods X are obtained as 300 kcal, 200 kcal, and 200 kcal, respectively, and are displayed in the band graph of the graph D20c at a rate of 3:2:2 in this order.

By displaying the graph D20b and the graph D20c with the same height, the type of the snack corresponding to the activity calorie consumed by the user is shown clearly. For example, since the hatched area of the zone B covers the shortcake area and reaches a middle of the ice stick area of the graph D20c, it is possible for the user to recognize the fact that the exercise corresponding to eating of the shortcake and a half of the ice stick has been performed. By performing such display, the consciousness of performing exercise in order to eat a desired snack is enhanced, and it can be expected to enhance the motivation of the user for continuing the exercise.

In the area D20g, there is displayed the generated message information of “WALK APPROX 90 MINUTES MORE.” This is a message for giving advice on the activity content corresponding to 300 kcal since the balance calorie of −300 kcal means that the activity calorie is 300 kcal smaller than the target calorie.

In order to display such message information, in the notification information generation section 41, the correspondence table having the information of the activity calorie per certain time for each of the types of the activity contents is stored (not shown) in the storage section 50, and the numerical value of the balance calorie is collated with the correspondence table to obtain the activity content and the activity time. In the example described above, the activity content is “walking,” and the activity time corresponds to “90 minutes.” Further, it is also possible to analyze the activity content inherent in the user actually performed by the user and the duration of the activity content based on the information obtained by the determination of the behavioral pattern and so on in the activity calorie calculation section 37 to thereby generate an original activity message based on the behavioral history of the user.

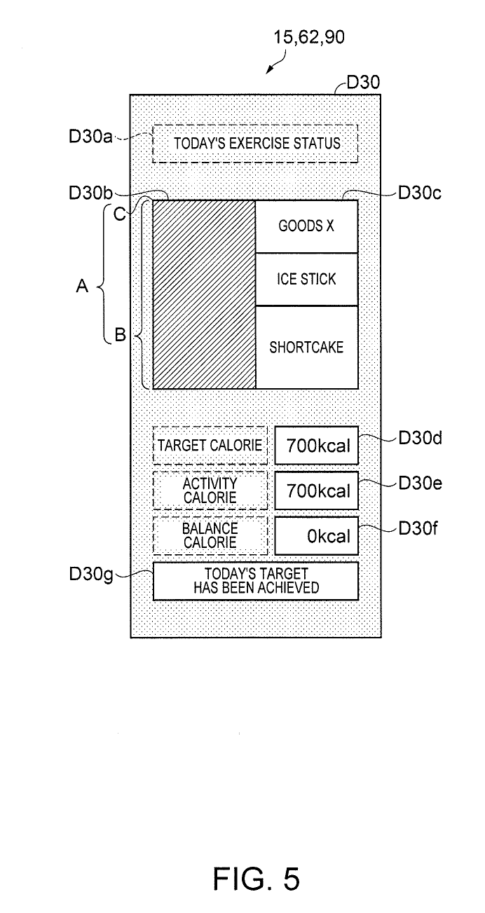

The notification information screen shown in FIG. 5 is a screen (the screen D30) displayed in the state in which the carry-over ON/OFF information is set to “OFF,” and the balance calorie is set to “0.” The screen D30 is a screen, which is displayed at the time point when the balance calorie becomes “0” after the user continues the activity from the state of the screen D20.

In the area D30a of the screen D30, there is displayed the same content as in the area D20a of the screen D20.

The graph D30b and the graph D30c of the screen D30 are substantially the same as the graph D20b and the graph D20c of the screen D20, respectively, and the graph D30c is the same in content as the graph D20c.

In the areas D30d, D30e, and D30f, there are shown that the target calorie is “700 kcal” (the area D30d), that the activity calorie is “700 kcal” (the area D30e), and that the balance calorie is “0 kcal” (the area D30f), respectively.

In the graph D30b, the hatched part of the zone B occupies the entire area of 100% with respect to the target calorie of the zone A. This shows the fact that the activity calorie of the exercise actually performed has reached the target calorie. The value of the zone C is 0% although the reference symbol is described alone.

In the area D30g, there is displayed the generated message information of “TODAY'S TARGET HAS BEEN ACHIEVED.”

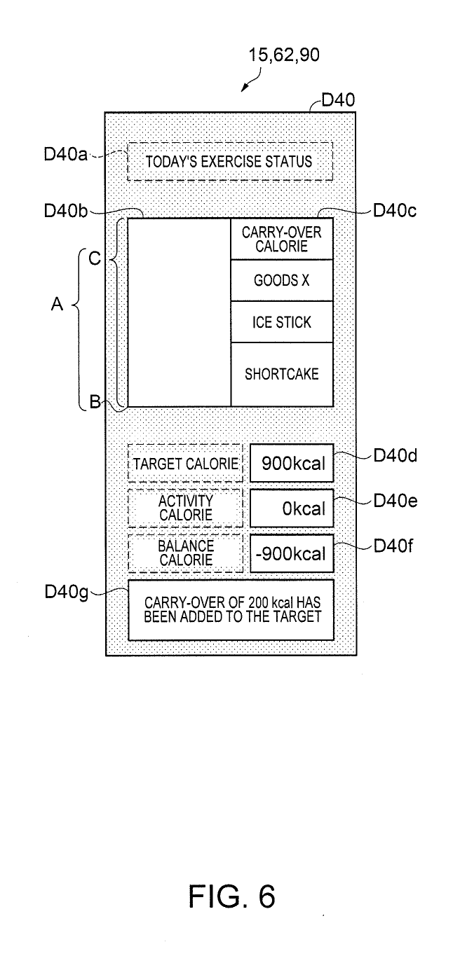

The notification information screen shown in FIG. 6 is a screen displayed in the state of starting the predetermined period, and is different from the notification information screens shown in FIG. 4 and FIG. 5 in the point that the carry-over ON/OFF information is set to “YES.” In that case, the carry-over calorie having been carried over is “−200 kcal.” Specifically, this is the case in which the activity calorie is 200 kcal smaller than the target calorie in the previous predetermined period, and therefore the calorie corresponding to 200 kcal is added at the start of the present predetermined period. For example, this corresponds to the case in which the yesterday's insufficient exercise is overcome today.

In the area D40a of the screen D40, there is displayed the same content as in the area D30a of the screen D30.

In the areas D40d, D40e, and D40f, there are displayed that the target calorie is “900 kcal” (the area D40d), that the activity calorie is “0 kcal” (the area D40e), and that the balance calorie is “−900 kcal” (the area D40f), respectively. In the breakdown of the target calorie of “900 kcal,” the carry-over calorie of 200 kcal is added to (−200 kcal is subtracted from) the sum of the shortcake of 300 kcal, the ice stick of 200 kcal, and the goods X of 200 kcal.

In the graph D40c, the breakdown of the target calorie is displayed with the items occupying the respective proportions. Specifically, the shortcake, the ice stick, the goods X, and the carry-over calorie are displayed at a rate of 3:2:2:2.

The graph D40b corresponds to the start point of the predetermined period, and is therefore displayed as an image showing the fact that the proportion of the target calorie is the zone A (zone B+zone C) of 100%, the proportion of the activity calorie is the zone B of 0%, and the proportion of the balance calorie is the zone C of 100%.

In the area D40g, there is displayed the generated message information of “CARRY-OVER OF 200 kcal HAS BEEN ADDED TO THE TARGET” to notify the user of the fact that the carry-over calorie has been added to the target calorie.

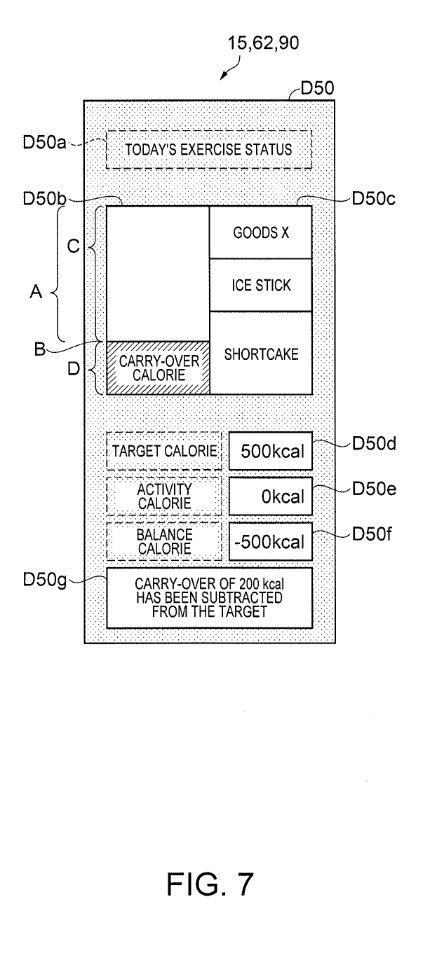

The notification information screen shown in FIG. 7 is a screen displayed in the state at the start of the predetermined period similarly to the screen shown in FIG. 6. Further, although the carry-over ON/OFF information is set to “ON” similarly to FIG. 6, the notification information screen shown in FIG. 7 is different in the point that the carry-over calorie having been carried over is “+200 kcal.” Specifically, this is the case in which the activity calorie exceeds the target calorie as much as 200 kcal in the previous predetermined period, and therefore the calorie corresponding to 200 kcal is subtracted at the start of the present predetermined period. This is applied to the case in which, for example, the exercise is reduced today as much as the excess in the yesterday's exercise to rest the body.

In an area D50a of the screen D50, there is displayed the same content as in the area D40a of the screen D40.

In the areas D50d, D50e, and D50f, there are displayed that the target calorie is “500 kcal” (the area D50d), that the activity calorie is “0 kcal” (the area D50e), and that the balance calorie is “−500 kcal” (the area D50f), respectively. In the breakdown of the target calorie of “500 kcal,” the carry-over calorie of 200 kcal is subtracted from the sum of the shortcake of 300 kcal, the ice stick of 200 kcal, and the goods X of 200 kcal.

In the graph D50c, the breakdown of the target calorie is displayed with the items occupying the respective proportions. Specifically, the shortcake, the ice stick, and the goods X are displayed at a rate of 3:2:2.

The graph D50b corresponds to an image shown at the start of the predetermined period, and shows the state in which the carry-over calorie has already been consumed at the start. The zone A of the graph D50b is the proportion of the target calorie, the zone B is the proportion of the activity calorie, the zone C is the proportion of the balance calorie, and the zone D is the proportion of the carry-over calorie. The zone A is a sum of the zone B and the zone C. The overall height of the graph D50b is a sum of the zone A and the zone D, and coincides with the overall height of the graph D50c. Specifically, the sum of the zone A (the target calorie of 500 kcal) and the zone D (the carry-over calorie of 200 kcal) corresponds to 700 kcal as a sum of the three types of snacks (the shortcake, the ice stick, and the goods X), and is shown with the same height.

In the state of the graph D50b, there is shown an image in which two thirds of the shortcake of 300 kcal corresponding to the carry-over calorie of 200 kcal has already been consumed before the user starts the exercise.

In the area D50g, there is displayed the generated message information of “CARRY-OVER OF 200 kcal HAS BEEN SUBTRACTED FROM THE TARGET” to notify the user of the fact that the carry-over calorie has been subtracted from the target calorie.

### Tallying Section

Going back to FIG. 2, the tallying section 43 has a function of tallying the information of the target calorie, the activity calorie, the balance calorie, the carry-over calorie, and the snacks straddling the predetermined periods in the medium and long terms. For example, the numerical values of each item are summed every certain period (e.g., one week, one month, or three months) . Further, the number of successful cases in which the activity calorie becomes equal to or higher than the target calorie is tallied in, for example, the predetermined period or a certain period. The total information thus tallied is stored (not shown) in the storage section 50. Further, it is also possible to deliver the information obtained by counting to the notification information generation section 41.

### Notification Information Generation Section/Message Information Of Giving Praise

Here, the function of the notification information generation section 41 generating the notification information based on the information tallied by the tallying section 43 will be described.

In the notification information generation section 41, the message information of praising the user is generated based on the total information delivered from the tallying section 43. The notification information generation section 41 determines whether or not the number of successful cases described above in a certain period has become equal to or larger than a predetermined number of times, and in the case in which it has become the predetermined number of times, the notification information generation section 41 generates the message information representing that the effort of the user is applauded and is praised. Further, it is also possible to perform a process of providing an amenity to the user in accordance with the content of the message information. In a specific example, in the notification information generation section 41, in the case in which the number of successful cases in a week (the certain period) becomes equal to or larger than the number of days (the predetermined number of times) of three days, such a message information as “the target is achieved for three days, a fun amenity is provided” is generated, and is output to the notification section 14. Further, the fun amenity is an amenity of, for example, applying the carry-over calorie to the next predetermined period to decrease the target calorie, or sending or transmitting a point or a badge for praising the user. It should be noted that the amenity is not limited to such an example, but a variety of amenities providing the user practical benefits can be applied.

The storage section 50 is formed of a storage device such as a read only memory (ROM), a flash ROM, a random access memory (RAM), a hard disk drive (HDD), or a solid state drive (SSD), and is configured including a working area (not shown) for temporarily storing the program 59 for realizing the functional sections of the processing section 30, a variety of programs, data, data in process and the process result of each of the functional sections, and so on.

The storage section 50 stores the snacking calorie correspondence table 51 described above, the target calorie data 52, the activity information data 53, the activity calorie data 54, the balance calorie data 55, the carry-over calorie data 56, and the program 59. Further, screen data 58 shows the fact that the screen data of the screens D20 through D50 described above are stored. It should be noted that the information stored in the activity information data 53 corresponds to the activity information.

### Server

The server 5 is constituted by an operation section 60, a display section 62, a communication section 64, a processing section 70, a storage section 80, and so on. The server 5 is a server installed on a network or in a cloud environment, and controls the information of the information apparatus 3 used by a plurality of users and a plurality of information apparatuses 7.

The operation section 60 is an input device such as a general keyboard or mouse, and the display section 62 adopts a liquid crystal panel as a preferable example. Further, the communication section 64 is a network adapter, and transmits and receives a variety of types of information related to snacking between the information apparatus 3 and the server 5.

The processing section 70 is a CPU, and integrally controls the sections of the server 5 such as the operation section 60, the display section 62, the communication section 64, and the storage section 80. The processing section 70 has a snacking calorie analysis section 71, a tallying section 73, and notification information generation section 75 as functional sections. It should be noted that these functional sections are illustrative only, and all of the functional sections are not necessarily essential constituents.

In the case in which the information of the snacking calorie corresponding to the snack information is requested by the snacking calorie acquisition section 33, the snacking calorie analysis section 71 searches of analyzes the snacking calorie of the snack information, and then responds the snacking calorie acquisition section 33 of the information apparatus 3 with the information of the snacking calorie thus obtained. In detail, the snacking calorie analysis section 71 accesses the snack information DB 81 managed in the storage section 80 (described later) to obtain the information related to the snack information managed on the database. In the case in which the same snack information exists, the snacking calorie analysis section 71 transmits the snacking calorie thereof to the information apparatus 3. In the case in which the snack information does not exist, the snacking calorie analysis section 71 refers to similar snack information in the snack information DB 81, and then transmits the alternative snack name and the snacking calorie thereof to the information apparatus 3. Further, the snacking calorie analysis section is capable of updating the information in the snack information DB 81 in each case in which the snacking calorie analysis section 71 has detected information related to a new type of snack while monitoring other servers and cloud servers on the Internet.

### Tallying Section

The tallying section 73 tallies the total information due to the activity of the user for each user. In detail, the tallying section 73 controls the communication section 64 to receive the total information from the information apparatus 3 used by the user. The tallying section stores the total information thus received in a user acquisition information DB 85 of the storage section 80 for each user. The total information is the total information tallied by the tallying section 43 of the information apparatus 3 described above, and further includes the information such as the activity information data 53.

### Notification Information Generation Section/Graphing Total Information Analyzed

The notification information generation section 75 analyzes the total information for each user to generate the notification information visualized by graphing or the like. In detail, in the case in which the notification information generation section 75 is required by the information apparatus or the information apparatus 7 to transmit the total information of the user, in the notification information generation section 75, the total information of the user stored in the user acquisition information DB 85 is read out, the screen data obtained by visualizing the variety of types of information related to the snacks to be eaten by the user and the activity for consuming the snacking calorie is generated, and then stored in the storage section 80 as screen data 88. Subsequently, the notification information generation section 75 transmits the screen data 88 in response to the transmission request from the information apparatus 3 or the information apparatus 7. The screen data 88 is output to the display section 15 of the information apparatus 3 or the display section 90 of the information apparatus 7, and is then displayed on the respective display device. Further, the screen data 88 is also output to the display section 62, and is also displayed on the display device of the server 5. FIG. 8 through FIG. 11 each show an example of the screen in the case in which the screen data 88 generated by the notification information generation section 75 is displayed.

FIG. 8 through FIG. 11 are diagrams each showing a graph screen of the total information.

The screen D100 (FIG. 8) is a screen of a bar graph in which the comparison between the activity calorie and the target calorie of the user is analyzed. In this graph, there is represented the comparison between the activity calorie and the target calorie in each of the days (December first through fifth).

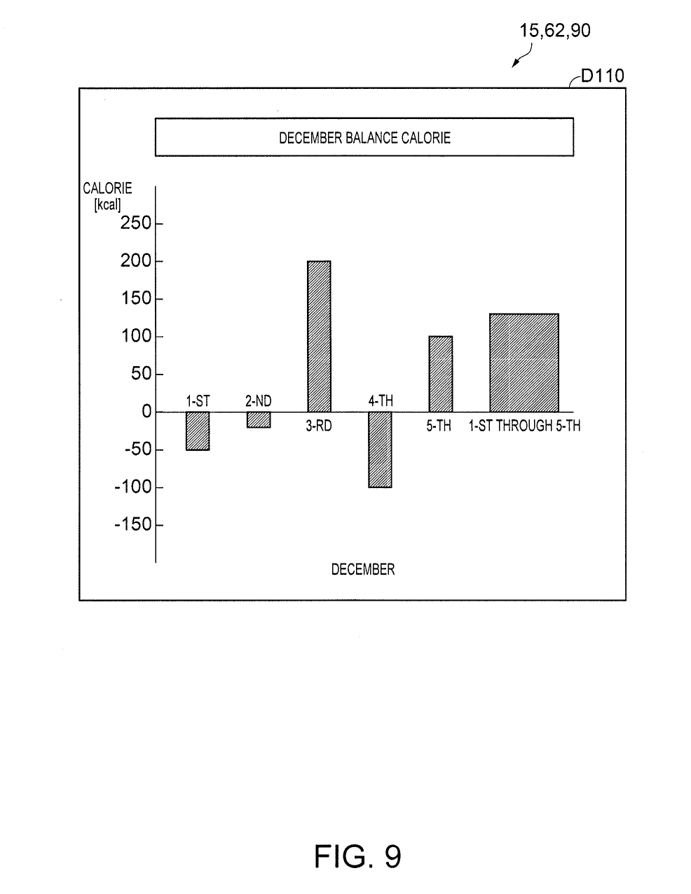

The screen D110 (FIG. 9) is a screen of a bar graph in which the balance calorie of the user is visualized. In this graph, there is represented the balance calorie ((the activity calorie)−(the target calorie)) in each of the days (December first through fifth). In “FIRST THROUGH FIFTH” in the right part of the screen D110, a sum of the balance calories in December first through fifth is represented with a bar graph.

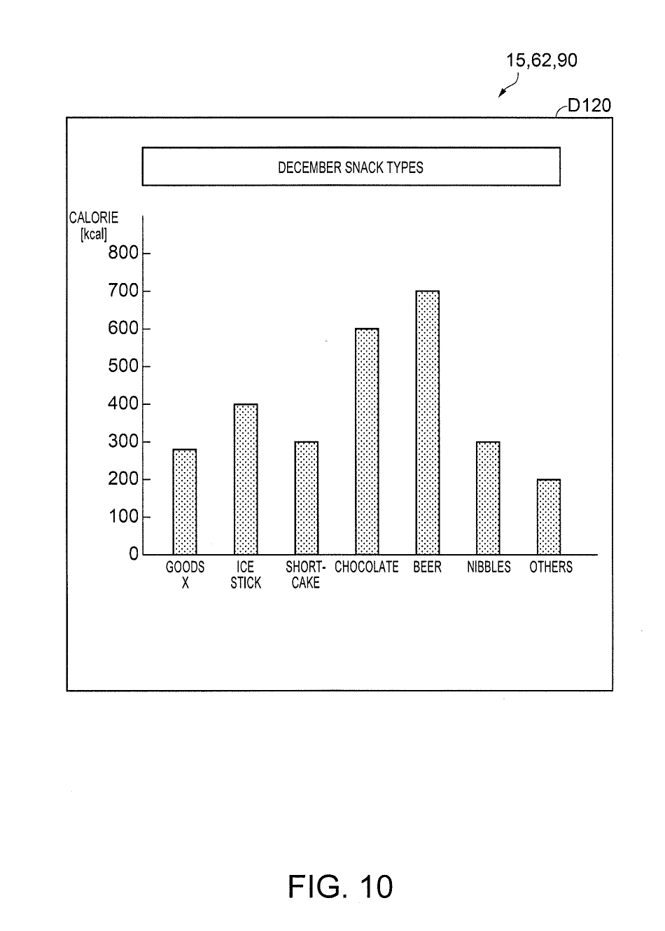

The screen D120 (FIG. 10) is a screen of a bar graph in which the snacking calorie of the snacks the user ate (or planed to eat) is analyzed type by type.

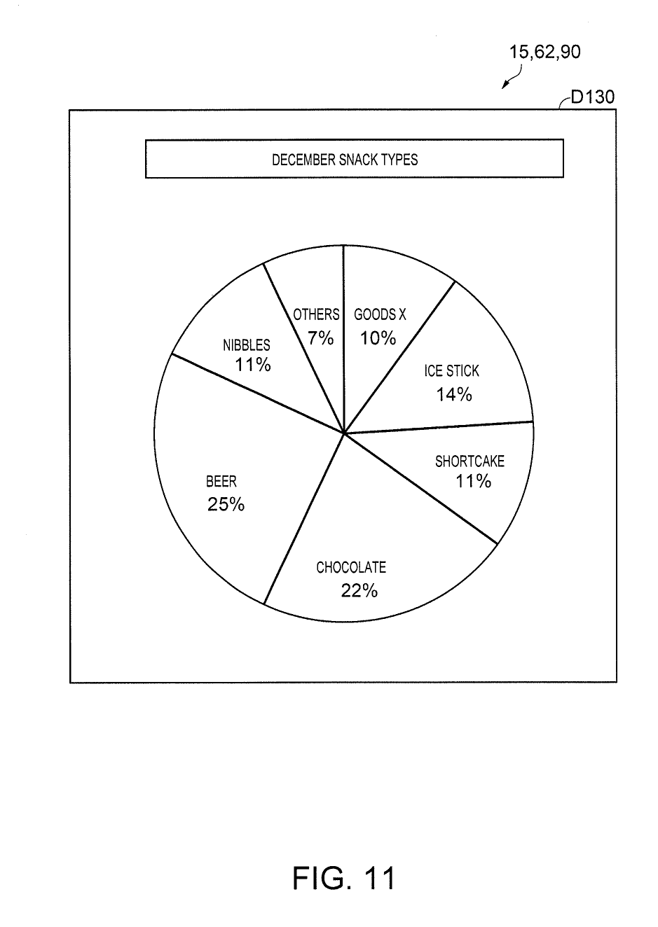

The screen D130 (FIG. 11) is a screen in which the information of the bar graph in the screen D120 is expressed by a pie chart.

It should be noted that the screen data 88 can also be generated in a format such as an HTML (hyper text markup language) format so as to be able to be displayed by a web browser or the like.

The storage section 80 is formed of a storage device such as a ROM, a flash ROM, a RAM, an HDD, or an SSD, and is configured including a working area (not shown) for temporarily storing the program 89 for realizing the functional sections of the processing section 70, a variety of programs, data, data in process and the process result of each of the functional sections, and so on. Further, the storage section 80 is equipped with a database engine such as a relational database, and there are performed the processes such as registration, update, deletion, and search of the data managed in the snack information DB 81, a user administrative DB 83, the user acquisition information DB 85 and so on, and a transaction process.

The snack information DB 81 is a database for managing information related to snacks, and stores the information such as snacking calorie, the unit of an amount of a snack, nutritional components of a snack, and cautions on ingestion type by type.

The user administrative DB 83 is a database for managing the personal information of the user, and stores an ID unique to the user, the name, the age, the sex, the weight, the height, and contact information of the user, apparatus information of the information apparatus 3 used by the user, family information of the user, apparatus information of the information apparatus 7 used by the family of the user, information related to the basic physical fitness of the user, and so on.

The user acquisition information DB 85 is a database for managing the total information received from the information apparatus 3 used by the user as described above, and accumulates the data such as the target calorie data 52, the activity information data 53, the activity calorie data 54, the balance calorie data 55, and the carry-over calorie data 56 user by user.

### Information Apparatus

The information apparatus 7 is a general PC, tablet PC, or the like, and is configured including the display section 90 as a second notification section. In the display section 90, there is displayed the screen data 88 received from the server 5 via the communication 4. Further, the information apparatus 7 is capable of obtaining the total information stored in the user acquisition information DB 85 of the server 5, and displaying the content equivalent to the screen data 58 displayed in the information apparatus 3. The information apparatus 7 is preferably an apparatus used by people involved in the user using the information apparatus 3, an advisor, or the like, and is capable of providing, for example, the family of the user with the information such as the screen data 58 and the screen data 88. Due to such a configuration described above, it is possible for the family of the user to check the information of the snacks the user plans to eat, and the information of the activity calorie. It is possible to realize such cooperation that the family of the user buys shortcake for the user and waits at home in the case in which the snack the user plans to eat is the shortcake, and the family of the user has confirmed the fact that the user has consumed calorie equivalent to the snacking calorie of the shortcake as the activity calorie.

### Snacking Control Method

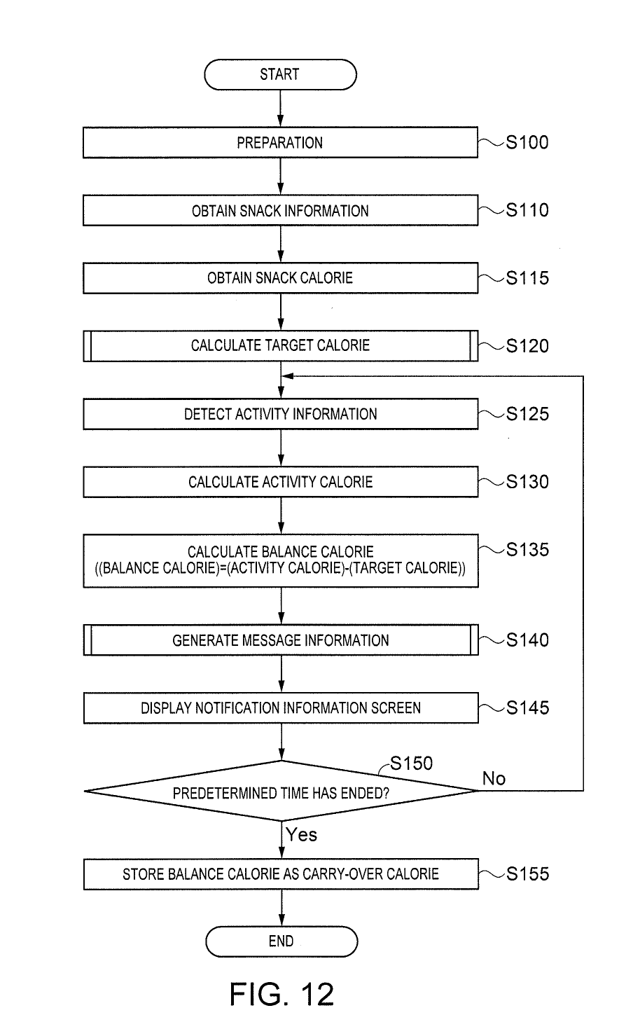

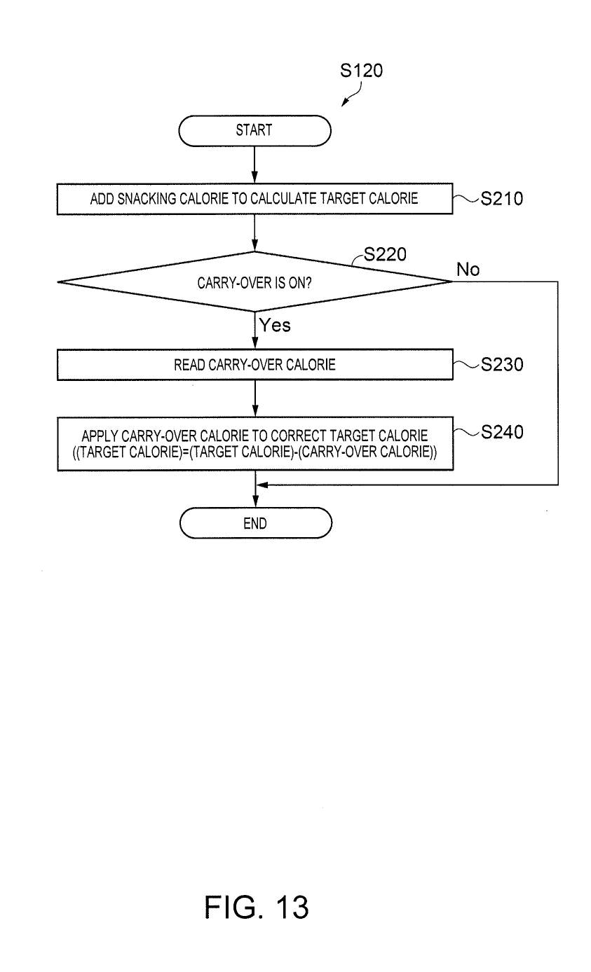

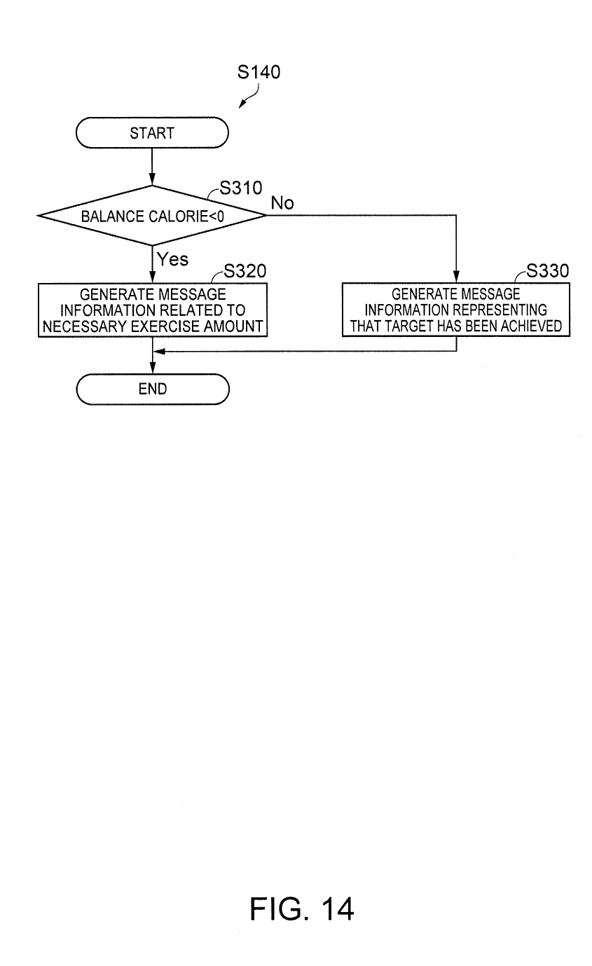

FIG. 12 is a flowchart showing a flow of a process of the snacking control. FIG. 13 is a flowchart showing a flow of a calculation process of the target calorie. FIG. 14 is a flowchart showing a flow of a generation process of the notification information. These flows are the flows of the processes performed by the processing section 30 of the information apparatus 3 and the processing section 70 of the server 5 reading in the program 59 and the program 89 respectively stored in the storage section 50 and the storage section 80, and then controlling the respective sections. It should be noted that the flows shown in FIG. 12 through FIG. 14 correspond to the snacking control method.

The snacking control method will be described using FIG. 12.

In the step S100, preparation is performed. Specifically, information (personal information stored in the user administrative DB 83) of the user using the information apparatus 3 and so on are obtained. The information thus obtained is stored in the storage section 50 of the information apparatus 3 and the user administrative DB 83. In particular, the age, the sex, and the weight of the user are used in the calculation of the activity calorie in the subsequent steps. Further, in the present step, the information of the predetermined period related to the period for controlling snacking, and the carry-over ON/OFF information are obtained from the user, and are stored in the storage section 50 of the information apparatus 3.

In the step S110, the snack information is obtained. In detail, the type of the snack and an amount of the snack are obtained from the input screen prompting input of the snack information. The present step corresponds to a snack information acquisition process.

In the step S115, the snacking calorie is obtained. In detail, the value of the snacking calorie is obtained by looking up the snacking calorie correspondence table 51 snack by snack. The present step corresponds to a snacking calorie acquisition process.

In the step S120, the target calorie is calculated. The details of the present step will be described later with reference to FIG. 13. The present step corresponds to a target calorie calculation process.

In the step S125, the activity information is detected. In detail, the activity information detected by the detection section 20 is obtained. The present step corresponds to a detection process.

In the step S130, the activity calorie is calculated. In detail, an amount of the metabolic heat (the calorie consumption) consumed in the state satisfying the predetermined conditions is calculated based on the activity information, and then the calorie consumption accumulated is calculated as the activity calorie. The present step corresponds to an activity calorie calculation process.

In the step S135, the balance calorie is calculated. Specifically, a value obtained by subtracting the target calorie from the activity calorie is obtained as the balance calorie. The present step corresponds to a balance calorie calculation process.

In the step S140, the message information is obtained. The message information is the information displayed in the message information display area (e.g., the area D20g (FIG. 4), the area D30g (FIG. 5)) of the notification information screen. The details of the present step will be described later with reference to FIG. 14. The present step corresponds to a notification information generation process.

In the step S145, the notification information screen is displayed. The notification information screen is, for example, the screen D20 (FIG. 4) or the screen D30 (FIG. 5). The graph D20c or the graph D30c is drawn using the snack information and the snacking calorie obtained in the steps described above. The areas D20d through D20f or the areas D30d through D30f are displayed, and the graph D20b or the graph D30b is drawn using the target calorie, the activity calorie, and the balance calorie calculated in the steps described above. Further, the message information generated in the step S140 is displayed in the area D20g or the area D30g. The present step corresponds to a notification process.

In the step S150, whether or not the predetermined period has ended is determined. In the case in which it has been determined that the predetermined period has ended (Yes), the process proceeds to the step S155, and in the case in which it has been determined that the predetermined period has not ended (No), the transition to the step S125 is made in order to obtain the next activity information.

In the step S155, the balance calorie is stored as the carry-over calorie. In detail, the value of the balance calorie obtained after the predetermined period has ended is stored as the carry-over calorie data 56 in the storage section 50 to terminate the present flow.

The snacking control method will be described using FIG. 13.

The present flow is a process flow of the process in the step S120 (calculation of the target calorie).

In the step S210, the snacking calories are added to each other to calculate the target calorie. In detail, the snacking calories of the respective snacks are added to each other, and the result is obtained as the target calorie.

In the step S220, whether or not the carry-over is active is determined. In detail, in the case in which the carry-over ON/OFF information obtained in the step S100 has represented “ON,” it is determined that the carry-over is active (Yes), and the process proceeds to the step S230. In the case in which the carry-over ON/OFF information has represented “OFF,” it is determined that the carry-over is not active (No), the present flow is terminated. In this case, the target calorie calculated in the step S210 is used as the target calorie in the step S125 and the subsequent steps.

In the step S230, the carry-over calorie is read in. In detail, the carry-over calorie data 56 stored at the end of the previous predetermined period is read in.

In the step S240, the target calorie is collected by applying the carry-over calorie. Specifically, the carry-over calorie read in in the step S230 is subtracted from the target calorie calculated in the step S210 to obtain the target calorie. The present flow is terminated, and the process proceeds to the step S125.

The snacking control method will be described using FIG. 14.

The present flow is a process flow of the process in the step S140 (generation of the message information).

In the step S310, whether or not the balance calorie is a negative value is determined. The fact that the balance calorie is a negative value represents the fact that the activity calorie is smaller in value than the target calorie, and therefore, the activity amount (the exercise amount) is smaller than the target calorie. In contrast, the fact that the balance calorie is no smaller than 0 represents the fact that the activity calorie becomes equal to or higher than the target calorie, and there represents the fact that the activity amount (the exercise amount) has reached the target calorie. In the case in which the balance calorie is a negative value (Yes), the process proceeds to the step S320, and in the case in which the balance calorie is no smaller than 0 (No), the process proceeds to the step S330.

In the step S320, the message information related to the necessary exercise amount is generated. In detail, the activity content and the activity time thereof necessary to consume the calories equivalent to the balance calorie are obtained. The message information of the activity content and the activity time thereof thus obtained is generated. The present flow is terminated, and the process proceeds to the step S145.

In the step S330, there is generated the message information representing that the target has been achieved. The present flow is terminated, and the process proceeds to the step S145.

As described hereinabove, in the snacking control system 1 according to the present embodiment, the following advantages can be obtained.

The snacking information acquisition section 31 obtains the information of the predetermined period and the snacks requested in the predetermined period. The target calorie calculation section 35 calculates a caloric value, which is necessary to consume the food caloric value corresponding to the snacks planned to be eaten in the predetermined period, as the target calorie based on the snacking calorie (the caloric information) obtained by the snacking calorie acquisition section 33. Subsequently, the activity calorie to be consumed due to the activity of the user is calculated by the activity calorie calculation section 37. Since the consumed caloric value due to the basal metabolism is not included in the activity calorie, by aggressively performing the activity (exercise), it is possible to consume the caloric value equivalent to the food caloric value of the snacks planned to be eaten before eating the snacks. The user is notified of the condition of the food caloric value of the snacks decreasing due to the exercise performed during the period with the notification information screen (the screen D20, the screen D30) generated by the notification information generation section 41. By visually recognizing such a notification information screen, the user can concentrate on the activity for consuming the target calorie corresponding to the snacks. In such a manner, the user is notified of an amount of the energy for the user to consume and an amount of the energy consumed in accordance with the activity of the user based only on the information related to the snacks as the information instead of the control including the meals as in the related art. Therefore, it is possible to provide a system placing a high value on the control related to snaking for the user eating snacks on a regular basis.

Further, the snacking information acquisition section 31 obtains the carry-over ON/OFF information. In the case in which the carry-over ON/OFF information is set to “ON,” if the activity (exercise) exceeding the target calorie has been performed in the predetermined period, the excess part of the activity calorie can be carried over to the subsequent predetermined period. By providing such a function of making the carry-over possible, it is possible to expect an advantage that the user can perform the activity (exercise) for the predetermined period with enthusiasm to enhance the motivation.

Further, in the information apparatus 7, it is possible to display a variety of notification information screens generated by the notification information generation section 75 of the server 5. It is also possible for the person involved in the user and helping the user reduce the weight to check a variety of notification information screens to support the activity (exercise) of the user. By being supported, the user can further increase the motivation for the weight reduction activity.

### Second Embodiment

### Wearable Device

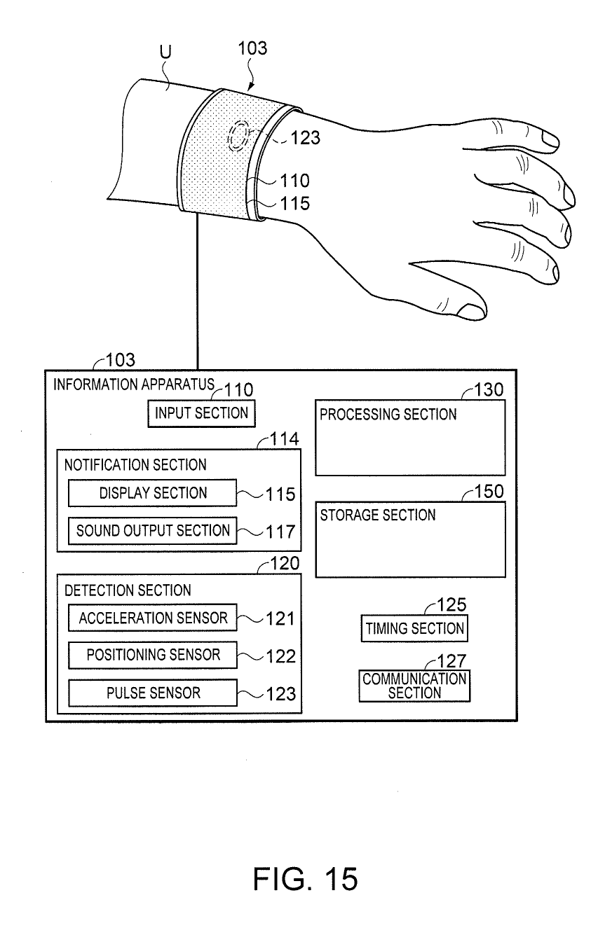

FIG. 15 is a diagram showing an outline of an information apparatus according to a second embodiment. A configuration of the information apparatus 103 according to the present embodiment will hereinafter be described. It should be noted that the same constituents as those of the snacking control system 1 according to the first embodiment are denoted by the same reference numerals, and the duplicated descriptions regarding the same constituents will be omitted. Further, although the server 5 and the information apparatus 7 explained in the description of the snacking control system 1 according to the first embodiment are not described in FIG. 15, it is also possible to adopt a configuration in which the server 5 and the information apparatus 7 are connected to each other by the communication 4. It should be noted that the information apparatus 103 corresponds to a wearable device.

The information apparatus 103 has substantially the same function as the information apparatus 3 of the snacking control system 1 according to the first embodiment, and is different from the information apparatus 3 in the point that the information apparatus 103 is the wearable device to be mounted on the user U.

The information apparatus 103 is a wristwatch-type device to be mounted on the wrist (or an arm part) of the user U. The information apparatus 103 is provided with a display section 115 exposed on a surface opposed to the wrist side in the state of being mounted on the wrist, and an input section 110 capable of inputting information using a touch panel system disposed so as to cover the display section 115. Further, there is provided a pulse sensor 123 disposed on the side surface of the skin of the wrist in the state of being mounted on the wrist.

In the block configuration diagram shown in FIG. 15, only principal constituents of the information apparatus 103 are described, and some of the constituents are omitted in the description. The information apparatus 103 is configured including the input section 110, a notification section 114 (the display section 115, a sound output section 117), a detection section 120 (an acceleration sensor 121, a positioning sensor 122, the pulse sensor 123), a timing section 125, a communication section 127, a processing section 130, a storage section 150, and so on, and is mounted on an MEMS (micro electromechanical systems) or a microchip miniaturized, reduced in height, and reduced in power consumption, and is installed in the wristwatch-type device main body.

Hereinafter, the display section 115, the input section 110 and the pulse sensor 123 as characteristic constituents of the information apparatus 103 will be described in detail.

The display section 115 is a display device capable of displaying a character, an image, and an icon, and is, for example, a flexible-type dot-matrix electrophoretic display (EPD) which can flexibly be deformed. The display section 115 performs a variety of types of display based on a display signal input from the processing section 130. It should be noted that the display section 115 can also be a liquid crystal display (LCD), a segment-type LED, or the like.

The input section 110 is an input device configured including a touch sensor, a touch panel, or the like covering the display surface of the display section 115, and detects the region having been touched using a method such as a capacitance method or a resistance film method, and then outputs a detection signal to the processing section 130. It should be noted that the input section 110 can also be an input device configured including a button switch and so on.

The pulse sensor 123 is formed of a photoelectric sensor provided with a light emitting element and a light receiving element, emits light from the light emitting element toward a living body, and receives the reflected light reflected by a blood vessel with the light receiving element. The pulse sensor 123 detects the pulse wave of the user using a phenomenon that the reflectivity of the light is different between the expansion process and the contraction process of a blood vessel. The pulse sensor 123 perform a frequency decomposition process on the data of the pulse wave thus detected to analyze the signal intensity value of each frequency to thereby identify the frequency spectrum corresponding to the pulse wave. Then, the pulse sensor 123 calculates the pulse rate based on the frequency of the frequency spectrum of the pulse wave. The pulse rate thus calculated is stored in the storage section 150. It should be noted that the pulse sensor 123 is not limited to the photoelectric sensor described above, but it is possible to adopt an ultrasonic sensor for detecting the contraction of the blood vessel using an ultrasonic wave to measure the pulse rate, or to adopt a sensor for making a weak current flow from an electrode through the body to measure the pulse rate, and so on. The pulse rate thus measured is stored in the storage section 150, and the activity calorie is calculated based on the information of the pulse rate by the processing section 130 using substantially the same method as the activity calorie calculation section 37 (FIG. 2) described above in the description of the first embodiment. It should be noted that the information of the pulse rate in the present embodiment corresponds to the activity information.

The processing section 130 controls the sections such as the input section 110, the display section 115, and the pulse sensor 123 to realize functional sections similar to the functional sections of the processing section 30 (FIG. 2) to thereby perform input of the snacks requested by the user, calculation of the target calorie, calculation of the activity calorie satisfying the predetermined conditions, calculation of the balance calorie, and generation of the notification information screen. The processing section 130 outputs the notification information screen to the display section 115, and thus, the screens D20 through D50, and the screens D100 through D130 (FIG. 4 through FIG. 11) are displayed on the display section 115.

As described hereinabove, according to the information apparatus 103, it is possible to realize the snacking control substantially the same as in the first embodiment with the wristwatch-type device to be mounted on the body of the user. Since the information apparatus 103 is mounted instead of the wristwatch of the user on a regular basis, a variety of types of activity information can be collected. Due to the variety of types of activity information, it becomes possible to accurately calculate the activity calorie of the user. Further, since the input section 110 and the display section 115 are provided, the notification information screen can visually be recognized in everyday life in the manner of visually recognizing the time of the wristwatch, and therefore, there can be expected enhancement of consciousness of the effort to perform the weight reduction activity of the user, and a maintenance and continuation effect.

It should be noted that the invention is not limited to the embodiments described above, but a variety of modifications and improvements can be provided to the embodiments described above. Some modified examples will be described below.

### First Modified Example

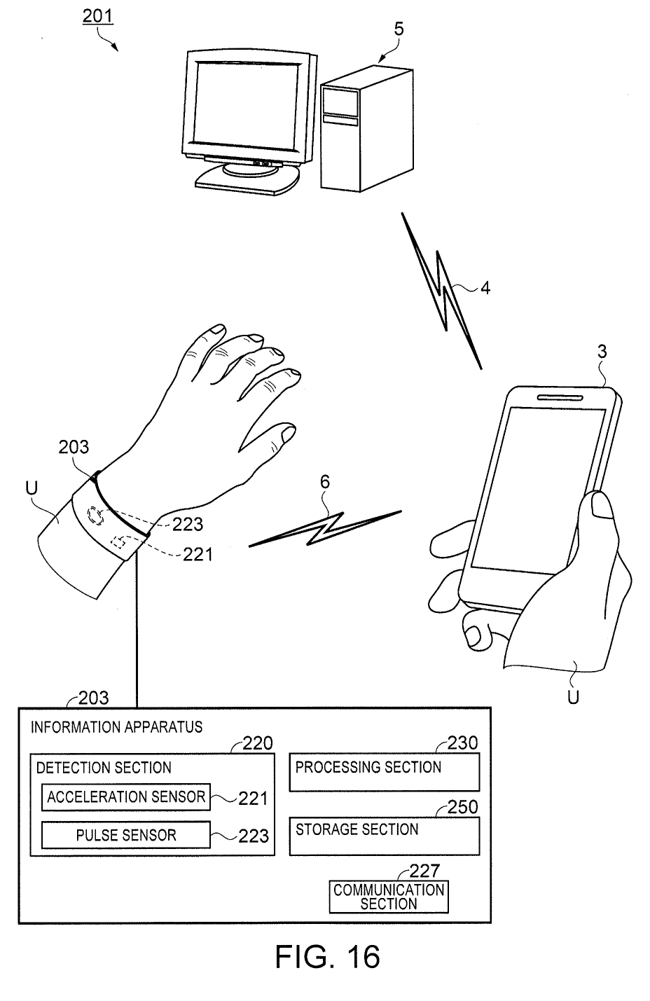

The first modified example will be described using the drawings such as FIG. 2 with a focus on FIG. 16.

FIG. 16 is an explanatory diagram showing an outline of a snacking control system in the modified example.

Although in the embodiments described above, there is adopted the configuration of detecting the activity information such as the acceleration information and the information of the pulse rate and then calculating the activity calorie using the information apparatus 3 or the information apparatus 103, the present modified example is different from the embodiments described above in the point that there is adopted a configuration of detecting the activity information using an information apparatus 203 separated from the information apparatus 3 or the information apparatus 103. It should be noted that the information apparatus 203 corresponds to a wearable device.

The snacking control system 201 is constituted by the information apparatus 203, the server 5, the information apparatus 3 and so on. The information apparatus 203 and the information apparatus 3 are connected to each other so as to be able to communicate a variety of types of data via communication 6, and the information apparatus 3 and the server 5 are connected to each other so as to be able to communicate a variety of types of data via the communication 4. It should be noted that in the snacking control system 201, it is also possible to adopt a configuration in which the information apparatus 7 shown in the first embodiment is connected to the server 5 via the communication 4.

The information apparatus 3 establishes communication with the information apparatus 203 via the communication 6 using the communication section 27 (FIG. 2) as a communication adapter. As a preferable example, the communication 6 corresponds to a near-field communication adapter. The activity calorie calculation section 37 of the information apparatus 3 receives the activity information from the information apparatus 203. The subsequent process of the activity calorie calculation section 37 is substantially the same as in the first embodiment, and the notification information screen thus generated is displayed on the display section 15. It should be noted that the information apparatus 3 can use both of the activity information obtained from the detection section 20 and the activity information received from the information apparatus 203, or can also adopt a method of selecting and then using either one of the activity information obtained from the detection section 20 and the activity information received from the information apparatus 203. For example, it is possible to adopted a configuration in which the activity calorie calculation section 37 obtains the activity information from the detection section 20 during the period in which the activity calorie calculation section 37 does not receive the activity information from the information apparatus 203.

The information apparatus 203 is a wristwatch-type device to be mounted on the wrist (or an arm part) of the user U similar to the information apparatus 103 according to the second embodiment. The information apparatus 203 is different from the information apparatus 103 in the point that the display device for displaying the notification information screen and so on is not provided. Therefore, when commercializing the information apparatus 203, since the display device is not provided, miniaturization, reduction in height, reduction in weight, reduction in power consumption, and so on can be achieved accordingly compared to the information apparatus 103.

The information apparatus 203 is constituted by a detection section 220 (an acceleration sensor 221, a pulse sensor 223), a communication section 227, a processing section 230, a storage section 250 and so on.

The processing section 230 has a function of transmitting the activity information obtained from the detection section 220 to the information apparatus 3. In detail, when the user U wears the information apparatus 203, the processing section 230 controls the detection section 220 to start the detection of the acceleration information and the information of the pulse rate by the acceleration sensor 221 and the pulse sensor 223. The processing section 230 stores the activity information such as the acceleration information and the information of the pulse rate thus detected in the storage section 250 in a time series manner.

Then, the processing section 230 controls the communication section 227 to detect whether or not the information apparatus 3 is located within the communication range and can communicate with the processing section 230. In the processing section 230, in the case in which it has been determined that the communication is available, the communication 6 is established with the communication section 27 of the information apparatus 3, and the activity information stored in the storage section 250 in a time series manner is transmitted.

Further, it is also possible for the processing section 230 to be provided with some of the functional sections included in the processing section 30 of the information apparatus 3. For example, in the processing section 230, there can be provided the activity calorie calculation section 37, the balance calorie calculation section 39, and a target calorie acquisition section (not shown) for obtaining the target calorie, which is calculated based on the snack information of the user U and the calorie information, should be consumed by the user U, from the information apparatus 3. According to such a configuration, the target calorie acquisition section obtains the target calorie from the information apparatus 3 via the communication section 227. In the processing section 230, the balance calorie, which is a difference between the activity calorie calculated in accordance with the activity of the user U and the target calorie. The processing section 230 establishes always-on communication using the communication section 227, the communication section 27 (the information apparatus 3), the near-field communication adapter, and so on, and transmits the balance calorie calculated in real time to the information apparatus 3. In the information apparatus 3, it is possible to display the balance calorie changing in accordance with the activity of the user U. Further, in this case, it is also possible to provide the information apparatus 203 with LEDs or the like low in power consumption, and perform the notification (e.g., red lighting, yellow lighting, and green lighting) corresponding to the level of the balance calorie thus calculated.

When commercializing the information apparatus 203 provided with such a configuration, since miniaturization, reduction in height, and reduction in weight can be realized, the wearing feeling is good when wearing the information apparatus 203, and the possibility that the user wears the information apparatus 203 at all times is increased. Since it is possible to obtain a larger amount of activity information of the user, it is possible for the information apparatus 3 to generate the notification information higher in accuracy.

### Second Modified Example

In the embodiments and the modified example described above, in the activity calorie calculation section 37, as the method of calculating the activity calorie obtained by accumulating the amount of the metabolic heat consumed in accordance with the activity satisfying the predetermined conditions, there are adopted the conditions based on the activity (exercise) high in exercise intensity, the pulse rate and the heart rate as the predetermined conditions, but the invention is not limited to such conditions. There can be adopted any conditions with which the activity (exercise) can be determined.

For example, in the case of using the pulse rate (the heart rate can also be adopted) as the activity information, it is also possible to determine that the predetermined conditions are satisfied in the case in which the pulse rate or the heart rate exceeds a predetermined pulse rate as the predetermined condition. The predetermined pulse rate can be replaced with a pulse rate at rest, a pulse rate during sleep, an ordinary pulse rate, or the like. Further, a rapid changing point of the pulse rate can be adopted as the predetermined condition. For example, it is possible to calculate the activity calorie in the case in which the pulse rate rapidly rises.

### Third Modified Example

The method of calculating the activity calorie obtained by accumulating the amount of the metabolic heat consumed in accordance with the activity satisfying the predetermined conditions in the activity calorie calculation section 37 is not limited to the method described above in the embodiments and the modified examples. For example, it is also possible to accumulate all of amounts of the metabolic heat including an amount of the basal metabolism, and calculate the value obtained by subtracting the caloric value corresponding the amount of the basal metabolism from the total amount of the metabolic heat thus accumulated as the activity calorie.

What is claimed is:

1. A snacking control system comprising:
a snacking information acquisition section adapted to obtain snack information requested by a user;
a snacking calorie acquisition section adapted to obtain calorie information corresponding to the snack information;
a target calorie calculation section adapted to calculate target calorie for the user to consume based on the snack information and the calorie information;
a detection section adapted to detect activity information as information of an activity of the user;
an activity calorie calculation section adapted to calculate activity calorie based on the activity information;
a balance calorie calculation section adapted to calculate balance calorie as a difference between the activity calorie and the target calorie; and
a notification section adapted to notify the user of notification information including the target calorie, the activity calorie, and the balance calorie.

2. The snacking control system according to claim 1, wherein
the notification information includes message information representing that a target has been achieved, in a case in which the activity calorie has become one of equal to and higher than the target calorie.

3. The snacking control system according to claim 2, wherein
the notification section notifies the user of notification information when a number of cases in which the activity calorie has become one of equal to and larger than the target calorie, and in a case in which the number of cases has become one of equal to and larger than a predetermined number of times.

4. The snacking control system according to claim 1, wherein
the notification information includes message information related to an activity for consuming the balance calorie in the notification information in a case in which the activity calorie is lower than the target calorie.

5. The snacking control system according to claim 1, further comprising:
a storage section,
wherein the snacking information acquisition section obtains a predetermined period as a period for calculating the balance calorie,
the activity calorie calculation section calculates the activity calorie in the predetermined period,
the balance calorie calculation section calculates the balance calorie in the predetermined period, and
the storage section stores the balance calorie at an end of the predetermine period as periodic balance calorie.

6. The snacking control system according to claim 5, wherein
the target calorie calculation section reads the periodic balance calorie from the storage section at a start of the predetermined period, and calculates the target calorie taking the periodic balance calorie into consideration.

7. The snacking control system according to claim 1, wherein
the snack information includes at least one of a name of food classification, a name of a product, and a name determined by a supplier as information related to a type, and at least one of a number, a weight, a proportion, and a volume as information related to a quantity for each type.

8. The snacking control system according to claim 1, wherein
the activity information includes at least one of a pulse rate, acceleration information, a heart rate, a breathing rate, and movement information.

9. The snacking control system according to claim 1, wherein
the activity calorie calculation section calculates the activity calorie based on a predetermined condition includes at least one of a condition for determining an exercise state in a determination on whether or not a state is the exercise state, a condition for determining activity metabolism in a determination on whether the activity metabolism is true or basal metabolism is true, and a condition for determining an acting state in a determination on whether or not the acting state is true.

10. The snacking control system according to claim 1, further comprising:
a tallying section adapted to generate total information obtained by tallying at least one of the snack information, the target calorie, the activity calorie, and the balance calorie for a evaluation period,
wherein the notification information generation section analyzes the total information to generate the notification information expressed as a graph.

11. The snacking control system according to claim 1, further comprising:
a second notification section adapted to notify an advisor giving advice to the user of the notification information.

12. A wearable device to be mounted to a body of a user comprising:
a snacking information acquisition section adapted to obtain snack information requested by the user;
a snacking calorie acquisition section adapted to obtain calorie information corresponding to the snack information;
a target calorie calculation section adapted to calculate target calorie for the user to consume based on the snack information and the calorie information;
a detection section adapted to detect activity information as information of an activity of the user;
an activity calorie calculation section adapted to calculate activity calorie based on the activity information;
a balance calorie calculation section adapted to calculate balance calorie as a difference between the activity calorie and the target calorie; and
a notification information generation section adapted to generate notification information including the target calorie, the activity calorie, and the balance calorie.

13. The wearable device according to claim 12, further comprising:
a notification section adapted to notify the user of the notification information.

14. A wearable device to be mounted to a body of a user comprising:
a target calorie acquisition section adapted to obtain target calorie, which has been calculated based on snack information requested by the user and calorie information corresponding to the snack information for the user to consume, and which the user should consume;
a detection section adapted to detect activity information as information of an activity of the user;
an activity calorie calculation section adapted to calculate activity calorie based on the activity information; and
a balance calorie calculation section adapted to calculate balance calorie as a difference between the activity calorie and the target calorie.

15. The wearable device according to claim 14, further comprising:
a communication section adapted to transmit information including at least the balance calorie to an information apparatus.

16. A snacking control method comprising:
obtaining snack information requested by a user;
obtaining calorie information corresponding to the snack information;
calculating target calorie for the user to consume based on the snack information and the calorie information;
detecting activity information as information of an activity of the user;
calculating activity calorie based on the activity information;
calculating balance calorie as a difference between the activity calorie and the target calorie; and
notifying notification information including the target calorie, the activity calorie, and the balance calorie to the user.

17. An information apparatus including a processing section adapted to execute a program recorded on a computer-readable recording medium, the program comprising:
obtaining snack information requested by a user;
obtaining calorie information corresponding to the snack information;
calculating target calorie for the user to consume based on the snack information and the calorie information;
detecting activity information as information of an activity of the user;
calculating activity calorie based on the activity information;
calculating balance calorie as a difference between the activity calorie and the target calorie; and
notifying notification information including the target calorie, the activity calorie, and the balance calorie to the user.
